(19) United States
(12) Patent Application Publication
(10) Pub. No.: US 2026/0115136 A1
(43) Pub. Date: Apr. 30, 2026

(54) Living Hydrogels and methods of making and using the same

(71) Applicant: The Penn State Research Foundation, University Park, PA (US)

(72) Inventors: Amir Sheikhi, University Park, PA (US); Roya Koshani, University Park, PA (US)

(73) Assignee: The Penn State Research Foundation, University Park, PA (US)

(21) Appl. No.: 19/165,850

(22) PCT Filed: May 6, 2025
(86) PCT No.: PCT/US2025/027893

§ 371 Date: Sep. 16, 2025

Related U.S. Application Data
(60) Provisional application No. 63/647,775, filed on May 15, 2024.

(51) Int. Cl. A61K 9/06; A61K 47/36; A61K 47/38

(52) U.S. Cl. CPC A61K 9/06; A61K 47/36; A61K 47/38

(57) ABSTRACT

Embodiments relate to acellular nanocomposite hydrogels exhibiting extracellular matrix (ECM)-like mechanics and self-healing properties. In particular, embodiments relate to nanocomposite hydrogels including network-forming biopolymers and anisotropic hairy nanoparticle linkers configured to convert the biopolymers to ECM-like analogues via ionic and dynamic covalent bonds. Embodiments further relate to method of forming the nanocomposite hydrogels and to methods of regenerating tissue including integrating the nanocomposite hydrogels with host tissues.

### CROSS-REFERENCE TO RELATED APPLICATIONS

This patent application is related to and claims the benefit of priority of U.S. Provisional Application 63/647,775, filed on May 15, 2024, the entire contents of which is incorporated by reference.

### FIELD

Embodiments relate to acellular nanocomposite hydrogels exhibiting extracellular matrix (ECM)-like mechanics and self-healing properties. In particular, embodiments relate to nanocomposite hydrogels including network-forming biopolymers and anisotropic hairy nanoparticle linkers configured to convert the biopolymers to ECM-like analogues via ionic and dynamic covalent bonds.

### BACKGROUND

Engineering dynamically responsive hydrogels that mimic the mechanical and biological properties of tissues may enable living soft materials. The majority of native mammalian tissues comprise non-living ECM and living cell ensembles. ECM dynamically respond to external physical stimuli and stiffen under shear. Accordingly, non-linear shear-stiffening mechanics are crucial for tissue structural support and cell functions, such as adhesion, migration, and differentiation.

Tuning biopolymer concentration has been the main approach to tailor the non-linear mechanics of ECM-mimetic soft materials, which may inevitably alter other hydrogel properties, including mechanical strength, swelling behavior, degradation rate, and biocompatibility. Additionally, synthetic hydrogels that mimic the ECM mechanical properties are undermined by the concerns associated with biocompatibility, immune response, and toxic degradation byproducts. As a mechanical-biological trait, self-healing is also highly desirable in fabricating living hydrogels to enable autonomous morphological and mechanical restorations following reoccurring deformation and defects.

### SUMMARY

Developing multifunctional living acellular hydrogels with ECM-like mechanics and self-healing properties remains an ongoing challenge. Most living tissues have hierarchical and anisotropic structures, partially arising from the multiscale three-dimensional (3D) organization of ECM constituents. The isotropy of bulk polymeric hydrogels limits their ability to faithfully replicate the structural and mechanical features of ECM. To address this shortcoming, nanoparticles have been included in 3D polymeric networks, yielding anisotropic nanocomposite hydrogels (NCH). Besides using anisotropic nanoparticles, nanoparticle-polymer crosslinking has also been tailored via non-covalent and/or covalent bond formation. Polymer-nanoparticle inclusions have widely been investigated in terms of microscale network dynamics and macroscale mechanical and rheological properties; however, a limited number of NCH with tunable ECM-mimetic mechanical characteristics, particularly strain-stiffening, exists. As an example, NCH with strain-stiffening and self-healing properties have been engineered via incorporating tannic acid-coated cellulose nanocrystals (CNC) into synthetic poly(vinyl alcohol)-borax dynamic networks. Other kinds of NCH with tunable strain-stiffening properties have been developed via manipulating the nature of polymer-nanoparticle crosslinks and density, albeit using synthetic polyisocyanide and anisotropic iron oxide nanorods.

It is believed that there has been no report of naturally derived NCH with anisotropic particles that mimic the dynamic characteristics of native ECM (i.e., strain-stiffening) or tissues (i.e., self-healing) and enable the control of these properties. Hairy CNC (HCNC) are a newly emerged member of the nanocellulose family, comprising a rod-like crystalline body with disordered cellulose chains (hairs) at both ends. The hairs contribute to the unique anisotropic surface chemistry and architecture. We hypothesize that chemically tailored HCNC, bearing a combination of carboxylate and aldehyde moieties on the hairs, can link polymer chains in NCH matrices via reversible ionic and covalent bonds, resulting in controlled strain-stiffening and self-healing properties.

Accordingly, we have developed acellular nanocomposite living hydrogels with non-linear mechanics and self-healing properties. A matrix biopolymer may be synthesized to provide two types of active sites—functional groups for both ionic and dynamic covalent bond formation. To impart dynamic properties to bulk hydrogels, we have also developed anisotropic bifunctional nanoparticle linkers. The network is engineered via varying the nanoparticle concentration and binding mechanisms (dynamic covalent bonding and/or ionic crosslinking).

In an exemplary embodiment, an acellular nanocomposite hydrogel comprises a biopolymer comprising a plurality of polymeric chains, wherein the polymeric chains have first active sites and second active sites; and a plurality of nanoparticle linkers, wherein the linkers comprise cellulose bodies having a plurality cellulose chains protruding from their ends, wherein a first portion of the cellulose chains are functionalized with first groups configured to bond to the first active sites, and wherein a second portion of the cellulose chains are functionalized with second groups configured to bond to the second active sites.

In some embodiments, the first groups are configured to covalently bond with the first active sites, and the second groups are configured to ionically bond with the second active sites.

In some embodiments, the first groups are aldehyde groups and the first active sites are hydrazides, such that the aldehyde groups and the hydrazides covalently bond to crosslink the linkers with the biopolymer.

In some embodiments, the second groups are carboxylate groups and the second active sites are carboxylate sites, such that the carboxylate groups are configured to ionically bond to the carboxylate sites to crosslink the linkers with the biopolymer.

In some embodiments, a portion of the nanoparticle linkers form linker bridges including linkers crosslinked to one another via one or more cations.

In some embodiments, the one or more cations include calcium ions.

In some embodiments, the first groups are configured to covalently bond with the first active sites, and the cations and/or the second groups are configured to ionically bond with the second active sites.

In some embodiments, the first groups are aldehyde groups and the first active sites are hydrazides, such that the aldehyde groups and the hydrazides covalently bond to crosslink the linkers with the biopolymer.

In some embodiments, the second groups are carboxylate groups and the second active sites are carboxylate sites, such that the carboxylate groups and/or the cations are configured to ionically bond to the carboxylate sites to crosslink the linkers with the biopolymer.

In some embodiments, the hydrogel exhibits shear-stiffening and self-healing properties.

In an exemplary embodiment, an acellular nanocomposite hydrogel comprises a dihydrazide-modified alginate biopolymer comprising a plurality of polymeric chains, wherein the polymeric chains have hydrazide active sites and carboxylate active sites; and a plurality of nanoparticle linkers, wherein the linkers comprise cellulose bodies having a plurality cellulose chains protruding from their ends, wherein a first portion of the cellulose chains are functionalized with aldehyde groups configured to bond to the hydrazide active sites, and wherein a second portion of the cellulose chains are functionalized with carboxylate groups configured to bond to the carboxylate active sites.

In some embodiments, a portion of the nanoparticle linkers form linker bridges including linkers crosslinked to one another via one or more cations.

In some embodiments, the linker bridges are configured to ionically bond to the carboxylate sites to crosslink the linkers with the biopolymer.

In an exemplary embodiment, a method of forming an acellular nanocomposite hydrogel comprises forming a plurality of nanoparticle linkers by oxidizing cellulose bodies to provide dialdehyde modified cellulose bodies, oxidizing the dialdehyde modified cellulose bodies to provide further modified cellulose bodies having a plurality cellulose chains protruding from their ends, wherein a first portion of the cellulose chains are functionalized with aldehyde groups, and a second portion of the cellulose chains are functionalized with carboxylate groups; mixing the further modified cellulose bodies with an ionic solution such that the second portion of the cellulose chains are also functionalized with cations; mixing the plurality of nanoparticle linkers with a biopolymer comprising a plurality of polymeric chains, having first active sites and second active sites, such that the aldehyde groups are configured to bond to the first active sites and the carboxylate groups and/or cations are configured to bond to the second active sites.

In some embodiments, the aldehyde groups are configured to covalently bond with the first active sites, and the carboxylate groups and/or the cations are configured to ionically bond with the second active sites.

In some embodiments, the first active sites are hydrazides such that the aldehyde groups and the hydrazides covalently bond to crosslink the linkers with the biopolymer.

In some embodiments, the second active sites are carboxylate sites such that the carboxylate groups and/or the cations are configured to ionically bond to the carboxylate sites to crosslink the linkers with the biopolymer.

In an exemplary embodiment, a method of regenerating tissue comprises integrating an acellular nanocomposite hydrogel with a host tissue, wherein the hydrogel comprises a biopolymer comprising a plurality of polymeric chains, wherein the polymeric chains have first active sites and second active sites; and a plurality of nanoparticle linkers, wherein the linkers comprise cellulose bodies having a plurality cellulose chains protruding from their ends, wherein a first portion of the cellulose chains are functionalized with first groups configured to bond to the first active sites, and wherein a second portion of the cellulose chains are functionalized with second groups configured to bond to the second active sites.

These and other embodiments shall be described in more detail herein and in the drawings that show exemplary embodiments. Therefore, other details, objects, and advantages will become apparent as the following description of certain present preferred embodiments thereof and certain present preferred methods of practicing the same proceeds.

### BRIEF DESCRIPTION OF THE DRAWINGS

The above and other objects, aspects, features, advantages, and possible applications of embodiments of the present innovation will be more apparent from the following more particular description thereof, presented in conjunction with the following drawings. Like reference numbers used in the drawings may identify like components.

FIG. 1 is an illustration showing an exemplary embodiment of nanocomposite hydrogels including biopolymers and nanoparticle linkers. The biopolymers may undergo ionic crosslinking and dynamic covalent hydrazone bond formation with bifunctional linkers.

FIG. 2 is an illustration showing proposed mechanisms governing the large deformation-induced stiffening of crosslinked hydrogels, involving (i) pulling out of stress-path undulations on polymer-polymer and polymer-linker networks, which is bending dominated, and (ii) stress path reorientation of biopolymer chains, which is dominated by stretching.

FIG. 3 is an illustration showing examples of human soft tissues, consisting of shear-stiffening ECM and cell ensembles conferring self-healing.

FIG. 4 is an illustration showing biopolymer synthesis via successive periodate oxidation and Schiff base reaction, along with the corresponding changes in ALG chemical structure. Note that the chemical structures are just representatives of functionalization.

FIG. 5 is a ¹H NMR spectra of ALG, DA-ALG, and DH-ALG, confirming the successful functionalization of ALG with aldehyde and hydrazide groups.

FIG. 6 is a graph showing aldehyde group content of DA-ALG, determined by the NaOH-titration of HCl released from the reaction of aldehyde groups with NH₂OH·HCl.

FIG. 7 is a graph showing aldehyde group content of DH-ALG.

FIG. 8 is a graph showing a conductometric titration curve of DH-ALG based on the titration of weak acid (i.e., carboxylate) using NaOH, yielding a carboxylate group content of 3.2 mmol g⁻¹.

FIG. 9 is a schematic illustration of nanoparticle linker synthesis via successive periodate-chlorite oxidation reactions, as well as the corresponding changes in cellulose chemical structure.

FIG. 10 is a graph showing a representative aldehyde titration curve of DAMC, obtained via an oxime formation method.

FIG. 11 is a graph showing a representative aldehyde titration curve of the linker.

FIG. 12 is a graph showing a representative carboxylate titration curve of the linker using a strong base (10 mM NaOH).

FIG. 13 is a graph showing ATR-FTIR spectra of cellulose fibrils, DAMC, and the linker.

FIG. 14 is an exemplary AFM image of nanoparticle linkers.

FIG. 15 is a table providing an overview of strain-stiffening and self-healing properties of conventional gels, comprising flexible chains, networks of semiflexible polymers, or ECM-mimetic biopolymer networks, compared with the inventive nanocomposite hydrogels. The inventive nanocomposite hydrogels mimic the ECM mechanics and are self-healing.

FIG. 16 shows graphs demonstrating the effect of time on the storage modulus of the nanocomposite hydrogels containing varying concentrations of linker (1.25 wt % (top-left), 1.75 wt % (top-right), 2 wt % (bottom-left), 2.25 wt % (bottom-right)) and Ca²⁺, at constant frequency=1 rad s⁻¹ and strain=0.1%. The tests were performed on gels ˜5 min after preparation.

FIG. 17 is a graph showing storage modulus (G′) versus oscillatory shear strain for the hydrogels, containing varying Ca²⁺-free linker concentrations. The matrix biopolymer (DH-ALG) concentration was 2 wt. %.

FIG. 18 shows the average pore size of hydrogels, containing (left) varying linker concentration (Ca²⁺ concentration=0 mM), or (right) varying linker concentrations at varying Ca²⁺ concentrations (0-36 mM), calculated using the storage modulus (G′) at constant strain=1% and frequency=1 rad s⁻¹. The pore size decreased by increasing the linker and/or Ca²⁺ concentrations. (n=3, *p<0.05, **p<0.01, ***p<0.001, ****p<0.0001).

FIG. 19 is a graph showing storage modulus (G′) versus oscillatory shear strain for the hydrogels, containing varying Ca²⁺ concentrations (1.25 wt. %) at a constant linker concentration and a constant frequency of 1 rad s⁻¹. The matrix biopolymer (DH-ALG) concentration was 2 wt. %.

FIG. 20 is a graph showing storage modulus (G′) versus oscillatory shear strain for the hydrogels, containing varying Ca²⁺ concentrations (1.75 wt. %) at a constant linker concentration and a constant frequency of 1 rad s⁻¹. The matrix biopolymer (DH-ALG) concentration was 2 wt. %.

FIG. 21 is a graph showing storage modulus (G′) versus oscillatory shear strain for the hydrogels, containing varying Ca²⁺ concentrations (2 wt. %) at a constant linker concentration and a constant frequency of 1 rad s⁻¹. The matrix biopolymer (DH-ALG) concentration was 2 wt. %.

FIG. 22 is a graph showing storage modulus (G′) versus oscillatory shear strain for the hydrogels, containing varying Ca²⁺ concentrations (2.25 wt. %) at a constant linker concentration and a constant frequency of 1 rad s⁻¹. The matrix biopolymer (DH-ALG) concentration was 2 wt. %.

FIG. 23 is a phase diagram of hydrogel behavior at varying linker and Ca²⁺ concentrations and a constant DH-ALG concentration (2 wt. %).

FIG. 24 is a graph showing differential modulus (K′) versus shear stress at a constant frequency of 1 rad s⁻¹ of hydrogels containing varying Ca²⁺-free linker concentrations.

FIG. 25 is a graph showing stiffening index (m) of hydrogels containing varying Ca²⁺-free linker concentrations.

FIG. 26 is a graph showing critical stress (σ_{c}) of hydrogels containing varying Ca²⁺-free linker concentrations.

FIG. 27 is a graph showing K′ versus oscillatory shear stress at a constant frequency of 1 rad s⁻¹, measured for the hydrogels containing varying Ca²⁺ concentrations and a constant linker concentration of 1.25 wt. %.

FIG. 28 is a graph showing K′ versus oscillatory shear stress at a constant frequency of 1 rad s⁻¹, measured for the hydrogels containing varying Ca²⁺ concentrations and a constant linker concentration of 1.75 wt. %.

FIG. 29 is a graph showing K′ versus oscillatory shear stress at a constant frequency of 1 rad s⁻¹, measured for the hydrogels containing varying Ca²⁺ concentrations and a constant linker concentration of 2 wt. %.

FIG. 30 is a graph showing K′ versus oscillatory shear stress for the hydrogels, containing 2.25 wt. % of linker loaded with varying Ca²⁺ concentrations. The Ca²⁺-loaded hydrogels were not strain-stiffening.

FIG. 31 is a graph showing the m of the hydrogels at varying Ca²⁺ (0-18 mM)-loaded linker concentrations (1.25-2.25 wt. %).

FIG. 32 is a graph showing the σ_{c} of the hydrogels at varying Ca²⁻ (0-18 mM)-loaded linker concentrations (1.25-2.25 wt. %).

FIG. 33 is a graph showing m-σ_{c} profiles of biological and synthetic gels, compared with the hydrogels containing 1.25 wt. % of nanoparticle linkers with (1) 9 mM of Ca²⁺ or (II) 18 mM of Ca²⁺, hydrogels containing 1.75 wt. % of nanoparticle linkers with (III) 0 mM of Ca²⁺, (IV) 9 mM of Ca²⁺, or (V) 18 mM of Ca²⁺, hydrogels containing 2 wt. % of nanoparticle linkers with (VI) 0 mM of Ca²⁺ or (VII) 9 mM of Ca²⁺, or (VIII) hydrogels containing 2.25 wt. % of nanoparticle linkers with 0 mM of Ca²⁺. PIC, DPB-PEG-P(AM-co-LAEMA), CC/DP, and BE-PEG stand for synthetic polyisocyanides, diphenylboronic acid-terminated telechelic poly-(ethylene glycol)-poly(acrylamide-co-2-lactobionamidoethylmethacrylamide), O-carboxymethyl chitosan-dibenzaldehyde-terminated telechelic-ploy-(ethylene glycol), and boronate ester-crosslinked 4-arm polyethylene glycol, respectively. Biological σ_{c} range ˜0.1-10 Pa.

FIG. 34 is a graph showing oscillatory shear strain amplitude tests at alternating low (1%)-high (500%) strain cycles conducted on the hydrogels containing linker concentration of 1.25 wt. %, loaded with 9 mM Ca²⁺.

FIG. 35 is a graph showing oscillatory shear strain amplitude tests at alternating low (1%)-high (500%) strain cycles conducted on the hydrogels containing linker concentration of 1.75 wt. %, loaded with 9 mM Ca²⁺.

FIG. 36 is a graph showing oscillatory shear strain amplitude tests at alternating low (1%)-high (500%) strain cycles conducted on the hydrogels containing linker concentration of 2 wt. %, loaded with 9 mM Ca²⁺.

FIG. 37 is a graph showing oscillatory shear strain amplitude tests at alternating low (1%)-high (500%) strain cycles conducted on the hydrogels containing linker concentration of 2.25 wt. % without Ca²⁺.

FIG. 38 is a graph showing oscillatory strain amplitude tests at alternating low (1%)-high (500%) strain cycles conducted on the hydrogels containing 1.25 wt. % of linker with 18 mM of Ca²⁺.

FIG. 39 is a graph showing oscillatory strain amplitude tests at alternating low (1%)-high (500%) strain cycles conducted on the hydrogels containing 1.75 wt. % of linker with 0 mM of Ca²⁺.

FIG. 40 is a graph showing oscillatory strain amplitude tests at alternating low (1%)-high (500%) strain cycles conducted on the hydrogels containing 1.75 wt. % of linker with 18 mM of Ca²⁺.

FIG. 41 is a graph showing oscillatory strain amplitude tests at alternating low (1%)-high (500%) strain cycles conducted on the hydrogels containing 2 wt. % of linker with 0 mM of Ca²⁺.

FIG. 42 shows representative photos that demonstrate the self-healing behavior of the nanocomposite hydrogels crosslinked with the linker (1.75 wt. %) covalently or with Ca²⁺ (9 mM)-loaded linker ionically and covalently (scale bar=1 cm).

### DETAILED DESCRIPTION

The following description is of exemplary embodiments and methods of use that are presently contemplated for carrying out the present invention. This description is not to be taken in a limiting sense, but is made merely for the purpose of describing the general principles and features of various aspects of the present invention. The scope of the present invention is not limited by this description.

Embodiments generally relate to acellular nanocomposite hydrogels exhibiting extracellular matrix (ECM)-like mechanics and self-healing properties. As the hydrogels mimic the mechanical and biological properties of tissues, the hydrogels enable living soft materials. For example, the hydrogels may mimic the mechanical and biological properties of natural ECM such that, once the hydrogels are integrated with host tissue, the hydrogels may provide a supportive environment to the tissue. More specifically, cells can migrate from surrounding host tissue into the hydrogels. Once cells are adhered to or otherwise integrated with the hydrogels, the cells may proliferate and enable tissue growth or regeneration.

Exemplary hydrogels may comprise network-forming biopolymers and anisotropic hairy nanoparticle linkers. The linkers are configured to crosslink with biopolymer chains via ionic and covalent bonds such that a three-dimensional biopolymeric network is formed.

Notably, the nanocomposite hydrogels may stiffen under shear. Such non-linear shear-stiffening mechanics may allow for tissue structural support and cell functions, such as adhesion, migration, and differentiation. The nanocomposite hydrogels may also be mechanically robust and preserve their integrity such that they may confer self-healing properties in a manner similar to the cell-imparted healing after tissue damage provided by natural ECM.

The anisotropic hairy nanoparticle linkers include hairy cellulose nanocrystals (HCNC). The HCNC include rod-like cellulose bodies, such as cellulose fibrils, crystals, or combinations thereof. The cellulose bodies have a plurality of semi-flexible cellulose chains (also referred to as “hairs”) protruding from or near their ends. The cellulose chains may be functionalized with one or more functional groups. The cellulose chains may be disordered such that they are configured to accommodate a high number of functional groups.

Functional groups may be selected from the group consisting of aldehydes, anionic groups (e.g., carboxylates, sulfates, sulfonates, phosphates), amines, hydrazides, thiols, epoxides, isocyanates, and mixtures thereof. The groups may be selected depending on factors such as the availability of reactive sites on the target biopolymer and/or the desired type of interaction(s) (e.g., covalent and/or non-covalent) between the target biopolymer and linkers.

In some embodiments, the cellulose chains may be functionalized with at least two different groups. For example, a first portion of the cellulose chains may be functionalized with a first group, a second portion of the cellulose chains may be functionalized with a second group, etc. The first group may be configured to bond to a first active site of the biopolymer, the second group may be configured to bond to a second active site of the biopolymer, etc. such that a network may be formed between the linkers and biopolymer.

In some embodiments, the cellulose chains may be functionalized with at least three different groups. For example, a first portion of the cellulose chains may be functionalized with a first group, a second portion of the cellulose chains may be functionalized with a second group, a third portion of the cellulose chains may be functionalized with a third group, etc. As will be described in further detail, the first group may be configured to bond to a first active site of the biopolymer, the second group may be configured to bond to a second active site of the biopolymer, the third group may be configured to bond to a third active site of the biopolymer, etc. such that a network may be formed between the linkers and biopolymer.

In some embodiments, the cellulose chains may be functionalized with at least four different groups. For example, a first portion of the cellulose chains may be functionalized with a first group, a second portion of the cellulose chains may be functionalized with a second group, a third portion of the cellulose chains may be functionalized with a third group, a fourth portion of the cellulose chains may be functionalized with a fourth group, etc. As will be described in further detail, the first compound may be configured to bond to a first active site of the biopolymer, the second group may be configured to bond to a second active site of the biopolymer, the third group may be configured to bond to a third active site of the biopolymer, the fourth group may be configured to bond to a fourth active site of the biopolymer, etc. such that a network may be formed between the linkers and biopolymer.

In some embodiments, the coexistence of more than one group functionalized on the cellulose chains may enable concurrent physical (ionic) and chemical (dynamic covalent) bond formation with the biopolymer. For example, the first group may be an aldehyde group configured to form a covalent bond with the biopolymer, and the second group may be an anionic group (e.g., carboxylate group) configured to form an ionic bond with the biopolymer. However, any group(s) configured to bond to active sites of the biopolymer may be used.

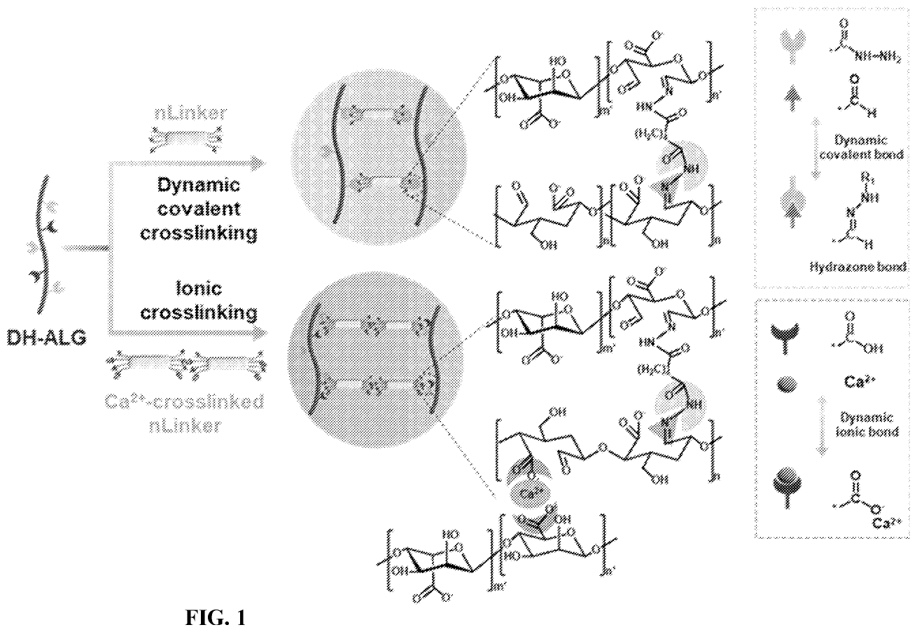

Ionic bonding between the anionic group and the biopolymer may be realized by using linker bridges. Linker bridges may be formed to help facilitate bonding between the linkers and biopolymers. Linker bridges may include more than one linker crosslinked to one another (see FIG. 1) via one or more ionic groups. In one embodiment, linkers may be modified with one or more cations configured to facilitate both inter-linker bonding (e.g., crosslinking between linkers) and inter-polymer bonding (e.g., crosslinking between polymer chains via linkers). The one or more cations may include Ca²⁺, Mg²⁺, Ba²⁺, Zn²⁺, Fe³⁺, Al³⁺, and mixtures thereof or any other suitable cation. The cations may be selected depending on factors such as the specific application, biocompatibility requirements, and/or desired binding strength. Moreover, the choice of cation can influence crosslinking density, reversibility, and/or environmental responsiveness of the final material.

In particular, the use of cations to crosslink the linkers may provide additional structural integrity and tunability to the overall network by introducing reversible, non-covalent ionic interactions. These cation-mediated linkages between functionalized linkers may enable a dual crosslinking system-covalent bonds ensure permanent network stability, while ionic crosslinks introduce dynamic, reversible behavior that may impart mechanical resilience, self-healing, or stimulus responsiveness of the material. While cation crosslinking may not be strictly necessary, it may offer functional advantages, particularly in applications where tunable mechanical or swelling properties are desired.

The biopolymer may be any type of polymer produced by living organisms. The polymer chains of the biopolymer may include one or more active sites configured to bond to and facilitate crosslinking with functional groups of the linkers.

In some embodiments, the biopolymer may include at least two different types of active sites. The biopolymer may include two different types of active sites, three different types of active sites, four different types of active sites, etc. For example, the first type of active site may be configured to bond to a first group of the linkers, the second type of active site may be configured to bond to a second group of the linkers, the third type of active site may be configured to bond to a third group of the linkers, the fourth type of active site may be configured to bond to a fourth group of the linkers, etc. The presence of multiple different types of active sites may advantageously enable concurrent physical (ionic) and chemical (dynamic covalent) bond formation between the biopolymer and linkers.

Any biopolymer that offers either ionic or covalent bonding functionality—or both—can be incorporated into this system. Examples include dihydrazide-modified alginate (DH-ALG), oxidized cellulose, chitosan (amine functionality), hyaluronic acid, gelatin (amine and carboxyl groups), carboxymethyl cellulose, and functionalized dextrans. These biopolymers can be tailored to interact with the linker components through ionic, hydrogen bonding, or dynamic covalent interactions.

In some embodiments, the biopolymer may be dihydrazide-modified alginate (DH-ALG). DH-ALG may provide two types of active sites, e.g., carboxylate groups and hydrazide for ionic and dynamic covalent bond formation, respectively. However, any biopolymer configured to provide one or more active sites may be used.

Referring to FIG. 1, in some embodiments, first active sites of the biopolymer may be configured to bond to aldehyde groups functionalized on the linkers. For example, the first active sites may include hydrazide, which is configured to bond to aldehyde groups. Such embodiments may enable chemical (dynamic covalent) bond formation between the biopolymer and linkers.

In some embodiments, second active sites of the biopolymer may be configured to bond to anionic (carboxylate) groups functionalized on the linkers. For example, the second active sites may include carboxylate groups, which are configured to bond to carboxylate groups functionalized on the linkers. Such embodiments may enable physical (ionic) bond formation between the biopolymer and linkers.

In some embodiments, one or more of the active sites may be configured to bond to linker bridges (e.g., more than one linker crosslinked to one another via one or more cations). For example, second active sites may include carboxylate groups, which are configured to bond to carboxylate groups and/or cations functionalized on linkers of the bridges.

Embodiments further relate to methods of making acellular nanocomposite hydrogels. Methods generally include a first step of producing the anisotropic hairy nanoparticle linkers. This first step may include at least a periodate oxidation step, and a chlorite oxidation step. In particular, the first step may include providing cellulose bodies, such as cellulose fibrils and/or crystals. The cellulose bodies may be derived from any suitable source. The cellulose bodies may be oxidized (e.g., with periodic acid or its salts) to synthesize dialdehyde modified cellulose (DAMC). The cellulose bodies may have vicinal diols (adjacent hydroxyl groups) in their structures, and this periodate oxidation step may cleave the carbon-carbon bond between the diols, resulting in the formation of aldehydes. This periodate step may be used to selectively oxidize the diols without affecting other functional groups present in the molecule. It is therefore contemplated that the DAMC may comprise reactive aldehyde groups that may be subject to further processing. The DAMC may further be oxidized (e.g., with chlorite ions) to synthesize DAMC modified with carboxylate groups. This second oxidation step may be carried out in acidic conditions and may result in the formation of chlorate ions as the primary oxidation product. In some embodiments, this step may be carried out such that half or approximately half of the reactive aldehyde groups of the DAMC are converted to carboxylate groups.

In some embodiments, the chlorite oxidation step may include adding DAMC to a solution comprising NaClO₂, and H₂O₂ may then be added (dropwise) to form DAMC modified with a desired amount of carboxylate groups.

Methods further include a step of combining (e.g., mixing) the formed linkers with biopolymers to form the hydrogels.

Methods may further include a step of forming linker bridges prior to forming the hydrogels. Linker bridges may be formed by combining (e.g., mixing) an ionic solution with a portion of the formed linkers.

Embodiments further relate to methods of using acellular nanocomposite hydrogels. For example, the hydrogels may facilitate tissue regeneration by integrating with host tissue. The hydrogels may provide a supportive environment to the tissue. More specifically, cells can migrate from surrounding host tissue into the hydrogels. Once cells are adhered to or otherwise integrated with the hydrogels, the cells may proliferate and enable tissue growth or regeneration.

In some embodiments, host tissue may be selected from the group consisting of soft and/or hard tissues, including nervous tissue (brain, spinal cord, nerves), epithelial tissue (skin, GI tract), muscle tissue (cardiac muscle, smooth muscle, skeletal muscle), and/or connective tissue (fat, bone, tendon, cartilage).

### EXAMPLES

### Materials

Bleached, delignified softwood kraft pulp sheets were provided by Resolute Forest Products Inc., Canada, and used as the starting material for the nanoparticle linker synthesis. Medium viscosity (MV) alginic acid sodium salt from brown algae was obtained from MilliporeSigma, USA. Sodium (meta)-periodate (NaIO₄, >99%), sodium chlorite (NaClO₂, 80%), sodium chloride (NaCl, >99.5%), hydrogen peroxide (H₂O₂, 30 wt. %), sodium hydroxide (NaOH, ACS reagent, >97%), hydrochloric acid (HCl, ACS reagent, 37%), ethylene glycol (Reagent plus, >99%), hydroxylamine-hydrochloride (NH₂OH·HCl, Reagent plus, 99%), adipic acid dihydrazide (ADH, NH₂NHCO(CH₂)₄CONHNH₂, ≥98%), calcium chloride dihydride (CaCl₂·2H₂O, ≥99%), poly-_{L}-lysin (PLL, 0.1 w/v % in water), Whatman® filter papers (grade 1, circles), and mineral oil (heavy) were purchased from MilliporeSigma, USA. AFM stainless steel disks and Mica sheets (V1 grade) were obtained from SPI supplies (USA) and Ted Pella Inc. (USA), respectively. Deuterium oxide (D₂O, 99.9%) was supplied by Cambridge Isotope Laboratories, Inc., USA. Anhydrous ethanol (200 proof) was purchased from KOPTEC, USA. All material synthesis and experiments were conducted using either Milli-Q water (resistivity ˜18.2 MΩ cm at T=25° C.) or deionized (DI) water, as specified.

### Nanoparticle Linker Synthesis

The linker was prepared according to the following method. The softwood pulp sheets (5 g) were torn into thin pieces with approximate dimensions of 2 cm×2 cm, and pulp fragments were soaked in DI water (500 mL) while stirring by an overhead stirrer (SH-II-6C, Faithful Instrument CO., China) overnight. Afterwards, water was removed via vacuum filtration using a nylon cloth (pore size: 20 μm), and the collected wet pulps were redispersed in a NaCl solution (325 mL, 1 M). Following 5 min mixing and thoroughly wrapping the beaker with aluminum (Al) foil, NaIO₄ (6.60 g) was added to selectively oxidize the vicinal diols of cellulose to dialdehyde groups. The reaction mixture was stirred at the ambient temperature for 42 h. Once completed, ethylene glycol (5 mL) was added and stirred for 10 min to deactivate unreacted NaIO₄. The oxidized pulps (dialdehyde modified cellulose, DAMC) were then vacuum filtered and rinsed with DI water at least 5 times (250 mL each time) to remove residual chemicals and byproducts.

To convert half of the DAMC aldehyde groups to carboxylate groups and isolate the linker, DAMC was further oxidized with NaClO₂. To this end, DAMC fibrils were suspended in DI water (250 mL, including the water in the wet DAMC), followed by adding NaClO₂ (4.22 g) and NaCl (14.12 g). H₂O₂ (4.22 g) was then added dropwise within 2 min after which the mixture was stirred at 100 rpm for 12 h. The pH was adjusted to 5.0±0.2 using a NaOH solution (0.5 M) during the reaction until pH remained almost constant at 5. The bifunctional fibrils were collected via centrifugation at 8,000 \g for 20 min and rinsed with an ethanol solution in DI water (70 v/v %, 100 mL) four times. The fibrils were suspended in DI water (500 mL) and heated at 80° C. for 2 h, followed by centrifugation at 8,000×g for 20 min to remove the unfibrillated cellulose. The linker particles in the supernatant were precipitated via adding a nonsolvent (ethanol, volume ˜1.5 times the supernatant volume), collected by centrifugation at 8,000×g for 20 min, and purified via dialysis (Spectra/Por membranes, M_{W} cutoff=8-10 kDa) against DI water for 1 day. The linker concentration was increased to 5 wt. % via evaporation at 40° C. under constant airflow for further use. The concentration was determined by oven-drying a known volume of linker dispersion at 60° C., followed by weighing.

### Matrix Biopolymer Synthesis

DH-ALG was synthesized via successive periodate oxidation and Schiff base reactions. A periodate-mediated oxidation reaction was conducted to functionalize alginate (ALG) with dialdehyde groups, yielding dialdehyde-modified alginate (DA-ALG, a reactive intermediate). DA-ALG synthesis was started by dispersing MV sodium ALG (5 g) in ethanol (50 mL), followed by adding DI water (200 mL). Ethanol as a non-solvent breaks up agglomerates, resulting in a homogenous distribution of ALG in water. Once fully dissolved, NaIO₄ (5.7 g) was added to the ALG solution in an Al foil-wrapped beaker while stirring by the overhead stirrer (speed=100 rpm) at room temperature. Given that NaIO₄ is a photosensitive oxidizer, the reaction was performed in the dark. After 1 h, the reaction was quenched by adding ethylene glycol (5 mL) and stirring for 10 min. To purify the resulting DA-ALG, NaCl (5 g) was added to the solution to screen the negatively charged carboxylate groups, facilitating precipitation. The DA-ALG was subsequently rinsed with an ethanol solution in DI water (70 v/v %, 800 mL each time) three times, and the precipitated DA-ALG was dissolved in DI water (250 mL). Further purification was conducted via dialysis (Spectra/Por membranes, M_{W} cutoff=8-10 kDa) against DI water for 1 day. To prevent microbial contamination, DA-ALG was stored at 4° C. until further use.

Next, the dialdehyde groups were reacted with hydrazide via a Schiff base reaction to form hydrazone bonds, yielding DH-ALG. ADH (0.9 g, ˜5 mmol) was dissolved in a DA-ALG solution (1 wt. %, 100 mL, ˜5 mmol aldehyde), and a Schiff base reaction was carried out for 5 h at room temperature. The ADH amount was calculated based on a 1:1 molar ratio of aldehyde group:ADH. The resulting DH-ALG solution was then dialyzed (Spectra/Por membranes, M_{W} cutoff=8-10 kDa) against DI water for 12 h. DH-ALG was stored at 4° C. and used within 3 days to minimize hydrazone bond hydrolysis.

### Nanocomposite Hydrogel Preparation

Nanocomposite hydrogels were formed via (i) linker-mediated dynamic covalent crosslinking of DH-ALG or (ii) simultaneous linker-mediated and ionic crosslinking of DH-ALG. The linker-crosslinked hydrogels (1 mL) were prepared via mixing a DH-ALG (5 wt. %) solution, the linker (5 wt. %) dispersion, and Milli-Q water at varying ratios to yield a constant final DH-ALG concertation of 2 wt. % and linker concentrations ranging from 1.25 to 2.5 wt. %. The biopolymer-linker hydrogels were mixed using a positive displacement pipette (Microman E M1000E, Gilson, OH, USA) for ˜30 s and maintained at room temperature for 5 min prior to any characterizations. To prepare the linker-ionically-crosslinked hydrogels, the linker (5 wt. %, 10 mL) dispersion was mixed with 0.0875 mL, 0.175 mL, or 0.350 mL of a CaCl₂) solution (5 M) to obtain a carboxylate:Ca²⁺ molar ratio of 1:0.5, 1:1, or 1:2, respectively, resulting in 4.95, 4.91, or 4.83 wt. % of final linker concentrations. After 5 min of incubation, Ca²⁺-loaded linker dispersions were mixed with the DH-ALG solutions (5 wt. %) to reach a total volume of 1 mL. The final DH-ALG concentration was maintained constant at 2 wt. %, and the final linker concentration was varied (1.25, 1.75, 2, or 2.5 wt. %) by adding Milli-Q water, similar to those in the linker-crosslinked hydrogels. Note that hydrogels with a 1:0.5, 1:1, or 1:2 molar ratio of carboxylate:Ca²⁺ are referred to as 9, 18, or 36 mM Ca²⁺, respectively. For the hydrogel preparation, Milli-Q water was used to dilute the linker dispersion when required.

### Material Characterization

Functional Group Content Measurement: Conductometric titration was performed to quantify the aldehyde (CHO) group content of DAMC, linker, and DH-ALG, as well as the carboxylate (COO⁻) group content of linker and DH-ALG using the automated Metrohm 907 Titrando (Metrohm AG, Switzerland). The concentration of aldehyde groups was measured via an oxime formation method. Briefly, a known amount of DAMC (50 mg), linker (30 mg), or DH-ALG (30 mg) was added to DI water (100 mL) while being magnetically stirred. The pH of DAMC suspension and a NH₂OH·HCl solution (20 mL, final concentration=5 wt. %) was independently adjusted to 3.5 using NaOH or HCl solutions. The oxime reaction was initiated by mixing the DAMC suspension and NH₂OH·HCl solution, producing HCl, which was titrated using a NaOH solution (10 mM) at a rate of 0.1 mL min-1. The endpoint was obtained when the pH was stabilized at the initial pH (3.5). The aldehyde group content was calculated based on the volume of NaOH consumed to neutralize the released HCl during the reaction of aldehyde groups with NH₂OH·HCl according to Equation (1).

$${C}_{CHO} = \frac{{V}_{NaOH} \times M}{{m}_{D A M C}} \qquad ( 1 )$$

where C_{CHO} is the aldehyde group content per unit mass of DAMC (mmol g⁻¹), V_{NaOH} is the NaOH volume (mL), Mis the molarity of NaOH (mM), and m_{DAMC} is the DAMC dry mass (g).

To measure the carboxylate group content, a known amount of linker, DH-ALG (20 mg dry mass) and NaCl solution (2 mL, 20 mM) were added to 100 mL of Milli-Q water. The pH of linker or DH-ALG dispersion was then decreased to 3.2 using a HCl solution (0.1 M). The dispersion was titrated by a NaOH solution (10 mM) up to PH˜11 at a titration rate of 0.1 mL min-1. The carboxylate group content was calculated based on the volume of NaOH required to neutralize the weak acid (i.e., carboxylate groups) using Equation (2).

$${C}_{{COO}^{-}} = \frac{{V}_{NaOH} \times N}{m} \qquad ( 2 )$$

where C_{COO}⁻ is the carboxylate content per unit mass of linker or DH-ALG (mmol g⁻¹), and m is the linker or DH-ALG dry mass (g).

Atomic Force Microscopy (AFM) Imaging: The morphology and dimensions of linker were investigated using AFM. Imaging was carried out on an atomic force microscope (Bruker Dimension Icon I, USA), equipped with a silicon nitride probe (Bruker ScanAsyst-Air, USA). The experiments were conducted using the PeakForce Tapping mode. To prepare AFM specimens, a freshly cleaved mica sheet (diameter=10 mm) was adhered to a stainless-steel disc (diameter=15 mm). The negatively charged mica was then covered with a PLL solution (0.1% w/v) to minimize the electrostatic repulsion between the linker and mica. After ˜15 min, the excess PLL was rinsed with Milli-Q water (100 μL) five times, followed by drying at room temperature for ˜30 min. Once dried, a small droplet of linker dispersion (˜10 μL, 0.1 wt. %) was deposited onto the mica sheet and left to dry overnight. The linker-coated mica sheet was then rinsed with Milli-Q water (100 μL) five times to eliminate any non-adsorbed particles or debris and air-dried prior to imaging. The acquired images were processed using the NanoScope Analysis software (Version 1.8, Bruker, accessed via Penn State Materials Characterization Lab, MCL). The linker length and width was determined via manually assessing over 50 particles using the Gwyddion software (Version 2.49, accessed via the MCL).

Attenuated Total Reflection-Fournier Transform Infrared (ATR-FTIR) Spectroscopy: The characteristic chemical bonds of linker, DA-ALG, DH-ALG, and nanocomposite hydrogels were identified using a FTIR spectrometer (Fisher scientific, USA) according to the Bouguer-Beer-Lambert law. The spectrometer was equipped with a single bounce diamond ATR accessory. Prior to the analysis, samples were frozen at −80° C., followed by sublimating the ice at 0.01 mbar overnight using a FreeZone benchtop freeze dryer (Labconco, USA). The dried materials were directly placed on the ATR crystal, and the maximum pressure was applied by lowering the pressure clamp while the incident angle was 45°. The spectra were obtained by averaging 100 scans in a wavenumber range of 4000 to 500 cm⁻¹ (resolution=6 cm⁻¹). The absorbance was obtained via referencing the spectrum of clean bare diamond and then converted to transmittance.

Hydrodynamic Size and ζ-potential Measurements: To determine the hydrodynamic equivalent size of linkers, an established method was followed. Measurements were carried out on a dynamic light scattering (DLS) instrument (Malvern Zetasizer Nano series, UK) at a fixed scattering angle of 90° and 25° C. The linker concentration was adjusted to 0.1% w/v using Milli-Q water, followed by pipetting 70 μL of it into a low-volume quartz cuvette (ZEN2112, Malvern, UK). The Z-average values, i.e., cumulants mean, were reported as hydrodynamic equivalent size.

To determine ζ-potential, the electrophoretic mobility of linker was measured using the Nano ZS Zetasizer (Malvern instrument, UK) at room temperature and pH=6.5. Before initiating the measurements, the linker dispersion was diluted to 0.1% w/v using Milli-Q water, followed by pipetting a desired volume (900 μL) into disposable folded capillary cells (Malvern, UK). To apply the effect of linker rod shape to the ζ-potential value, Oshima's mobility expression was used. Since the κa˜1 (κ is the Debye-Hückel parameter, and a is the rod-like linker width, ˜5 nm, obtained from the AFM image analysis), the Henry's function ƒ (κa)˜0.5, and the following equation was used to calculate the ζ-potential using the electrophoretic mobility. Note that the ionic strength was calculated by considering the counter ions (Na⁺) of linker carboxylate groups. (see Equation (3)):

λₐᵥ=μ_{∥}+2μ_{⊥}/3=εᵣε₀/3η[1+2ƒ(κa)] (3)
where μ_{∥} and μ_{⊥} are the electrophoretic mobility of cylindrical particles in the parallel and perpendicular direction to the electric field, respectively, εᵣ denotes the relative dielectric permittivity of solvent, ε₀ is the dielectric permittivity in a vacuum (vacuum permittivity), and η denotes the electrolyte solution viscosity.

Proton (¹H) Nuclear Magnetic Resonance (NMR) Spectroscopy: To verify and quantify the aldehyde and hydrazide groups on DH-ALG, 1H NMR spectroscopy was performed using an NMR instrument (Bruker Avance III, MA, USA), operated at 600 MHz and equipped with a triple resonance inverse (TCI) cryoprobe. Freeze-dried ALG, DA-ALG, or DH-ALG (15 mg) were separately dissolved in D₂O (1 mL) for 2 h at room temperature. The spectra were acquired via averaging a total of 128 scans. The characteristic peaks were assigned to the respective functional groups. The area under the corresponding peaks, which was proportional to the number of hydrogen atoms, was measured using TopSpin software (Bruker, Version 4.0.7).

Rheological Analysis: Rheological properties were measured using an AR-G2 rheometer (TA Instruments, DE, USA), equipped with a parallel plate geometry with an upper plate diameter of 20 mm and a truncation gap of 1000 μm. Freshly prepared nanocomposite hydrogels were sandwiched between two parallel plates. After trimming the excess hydrogel, the air-exposed area was covered with a mineral oil to prevent water evaporation from the gel during the experiments. To assess the strain-induced stiffening of nanocomposite hydrogels, an oscillatory amplitude sweep test was conducted at a constant frequency of 1 rad s⁻¹ while varying the strain from 0.1 to 200%. To investigate the self-healing of nanocomposite hydrogels, recovery tests were conducted, wherein the dynamic moduli (i.e., storage modulus, G, and loss modulus, G″) were measured by applying alternating strain of 1% and 500% at time intervals of 100 s and a constant oscillation frequency (1 rad s¹). The time sweep test was performed on freshly prepared hydrogels at a constant frequency of 1 rad s⁻¹ and strain of 0.1% for 15 min. Temperature was maintained at ˜20° C. in all measurements.

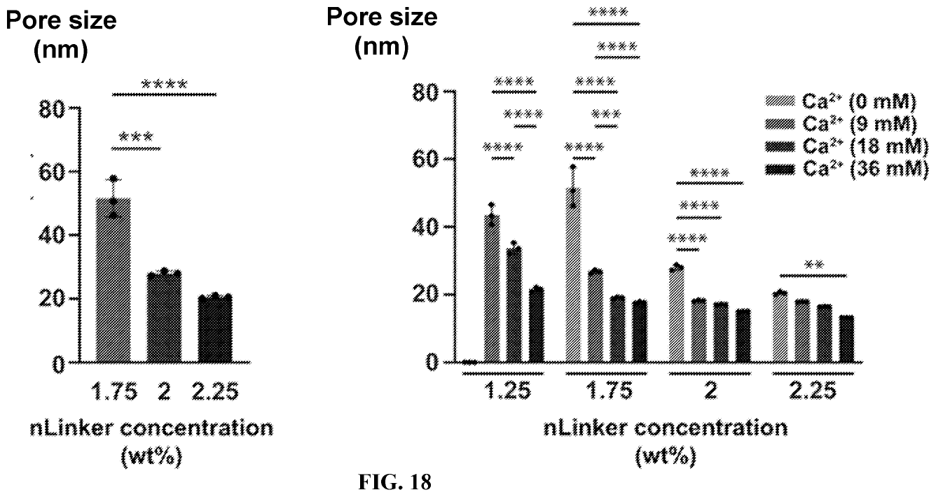

Statistical Analysis: Data were reported as the average of three independent samples (n=3)±standard deviation (SD), unless otherwise specified. GraphPad Prism (Version 9.4.1, MA, USA) was used for all statistical analyses. One-way (FIGS. 18, 25, and 26) or two-way (FIGS. 18, 31, and 32) analysis of variance (ANOVA), followed by the Tukey post-hoc test was conducted to determine the statistical significance. The p-values<0.05 were considered statistically significant (*p<0.05, **p<0.01, ***p<0.001, ****p<0.0001).

### Results

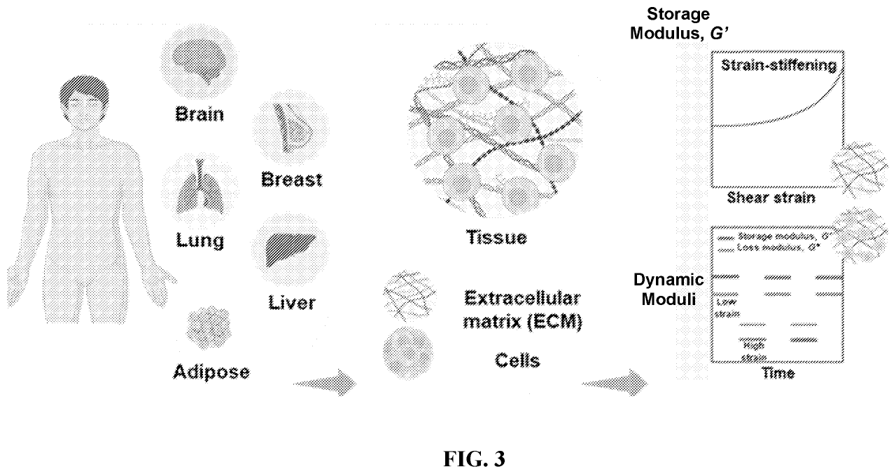

Hydrogel Design: FIG. 3 shows examples of human soft tissues and their main components, including ECM and cell ensembles, which have strain-stiffening and self-healing properties, respectively. The hydrogels were prepared via incorporating the bifunctional particle in reactive DH-ALG, prepared from semi-flexible ALG, as shown in FIG. 1. ALG, the matrix biopolymer of the hydrogels, has a semi-flexible backbone with carboxylate groups and vicinal diols in repeating units, allowing for NaIO₄-mediated selective oxidation and further functionalization with ADH (i.e., DH-ALG formation). The particle contains carboxylate and aldehyde groups, which are able to undergo Ca²⁺-mediated ionic crosslinking and Schiff base formation with the carboxylate and hydrazide groups of DH-ALG, respectively. The particle may enable not only dynamic covalent bonding (i.e., hydrazone bonds) with DH-ALG through a Schiff base reaction but also nanoparticle-polymer assembly via the Ca²⁺-mediated bridging of carboxylate groups in the DH-ALG matrix.

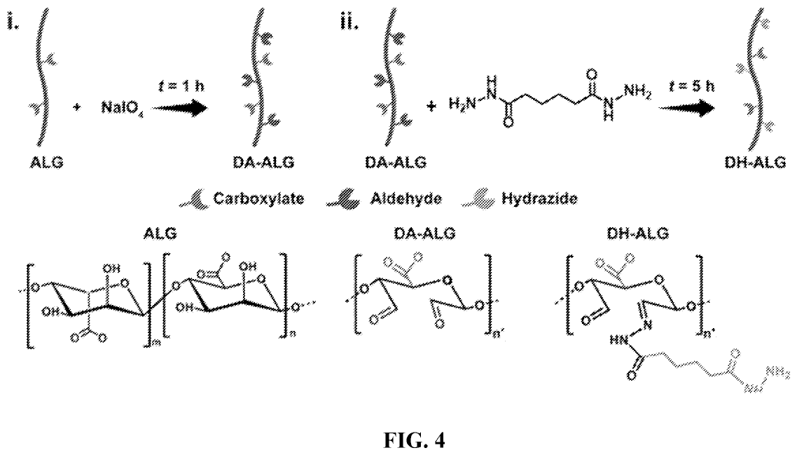

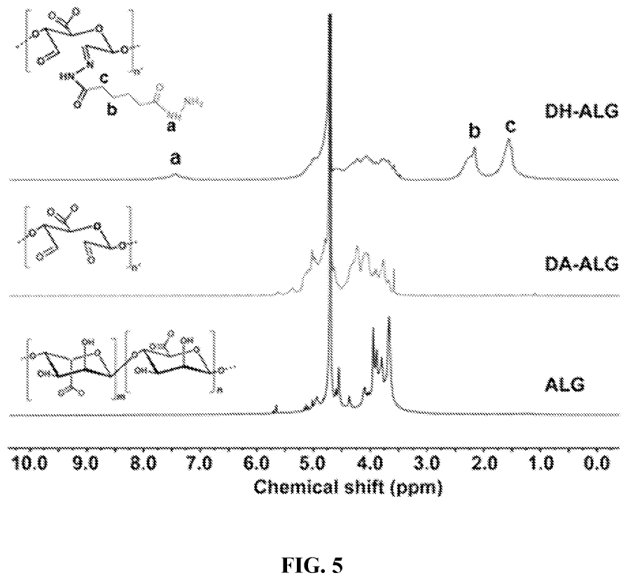

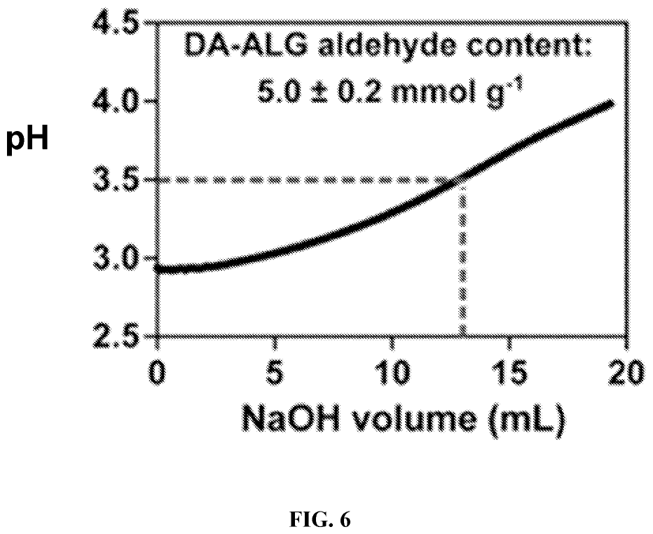

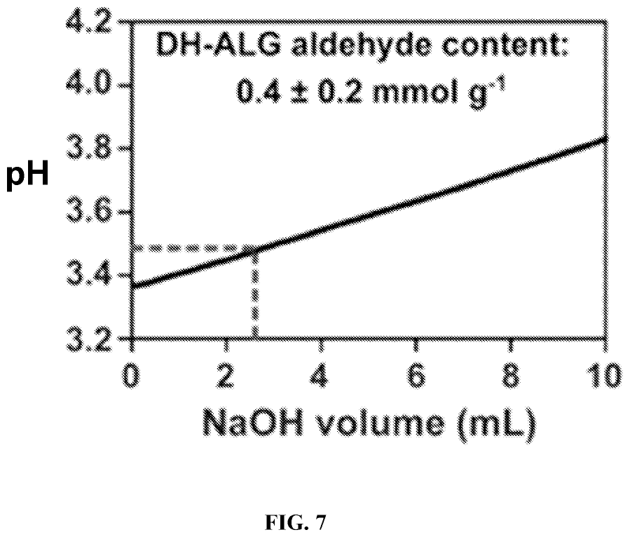

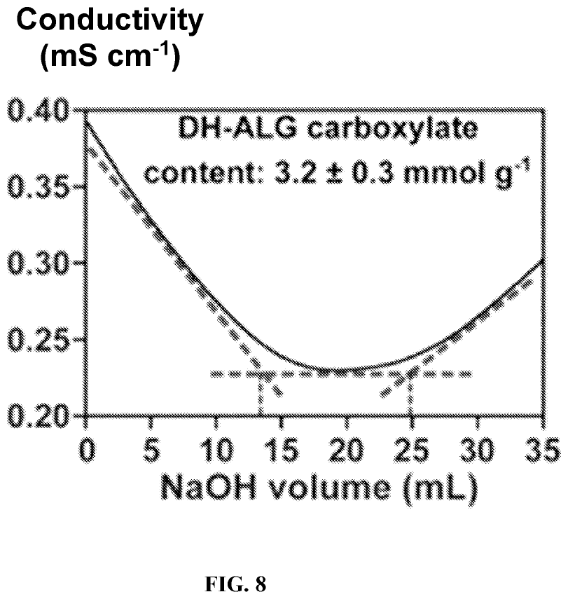

The chemical modification of ALG is schematically shown in FIG. 4. The vicinal diols are converted to aldehyde groups via NaIO₄-mediated oxidation, yielding DA-ALG (FIG. 4, left). Aldehyde groups are then converted to hydrazide via a Schiff base reaction, yielding DH-ALG (FIG. 4, right). Hydrazides are commonly used for developing dynamic covalent bonds with aldehyde groups. ALG chemical modification is assessed via ¹H NMR spectroscopy, and the spectra of ALG, DA-ALG, and DH-ALG are shown in FIG. 5. In the DA-ALG spectrum, two discrete peaks at the chemical shifts of ˜5.35 ppm and 5.63 ppm were associated with the aldehyde groups in their hemiacetal forms. Aldehyde groups of DA-ALG (5.0±0.2 mmol g⁻¹, FIG. 6), are reacted with hydrazide to form DH-ALG. In the DH-ALG spectrum, peak (a) ranging from 7 to 8 ppm corresponds to the —NH—, originated from the Schiff base reaction of DA-ALG dialdehyde groups with the hydrazide. In addition, peaks (b) and (c) at 1.5 ppm and 2.25 ppm, respectively, are attributed to the —CH₂— groups. FIG. 7 presents the content of remaining DH-ALG aldehyde groups after the reaction with hydrazide, which was only 0.4±0.2 mmol g⁻¹. Additionally, the carboxylate group content of DH-ALG is 3.2±0.3 mmol g⁻¹ (FIG. 8), which agrees with the previously reported values (e.g., 3 mmol g⁻¹) for alginic acid.

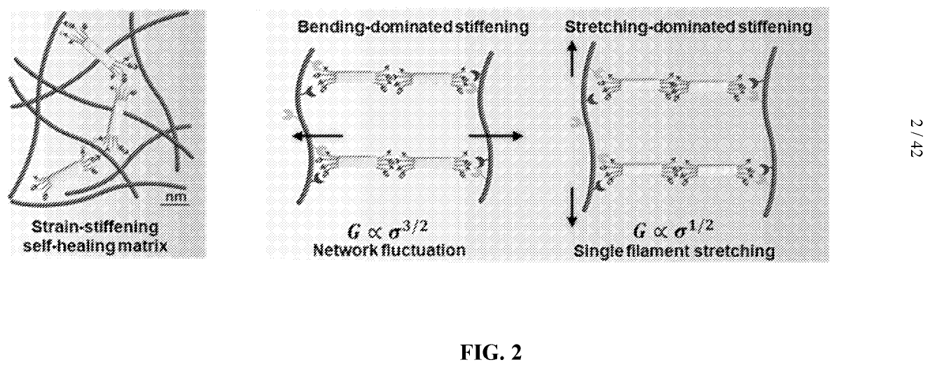

The schematic of DH-ALG/linker networks within the hydrogel matrix is presented in FIG. 2, indicating linker/linker and DH-ALG/linker crosslinks. FIG. 2 also shows proposed mechanisms governing the strain-stiffening behavior of these crosslinked networks. In silico studies on random networks comprising crosslinked biopolymer chains have shown that a large-strain response is followed by the development of a stress path for axially stressed fibers and crosslinks. Strain-stiffening in the hydrogel may be resulted from two mechanisms: (i) pulling out of stress-path undulations on DH-ALG/DH-ALG and DH-ALG/linker conjunctions and (ii) the reorientation of stress path on DH-ALG. The pulling out of stress-path undulations is bending-dominated in which case shear modulus (G) is correlated with stress (σ) to the power of 3/2, mainly resulting from the entropic behavior of biopolymers. When the biopolymer chains are crosslinked within a network, the interplay between the entropic behavior of chains and the constraints imposed by the crosslinks (i.e., network fluctuations) may lead to nonlinear mechanical behaviors. As σ increases, the polymer network becomes stiffer because of the entropic effects of biopolymer chains. The reorientation of stress path is stretch dominated in which case G is correlated with σ to the power of ½, originating from single filament stretching.

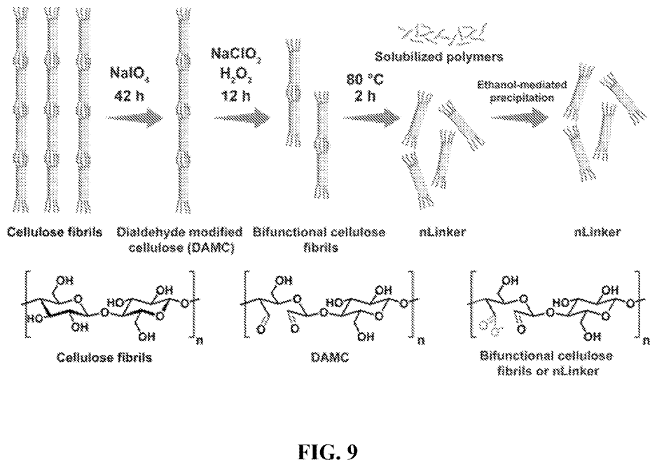

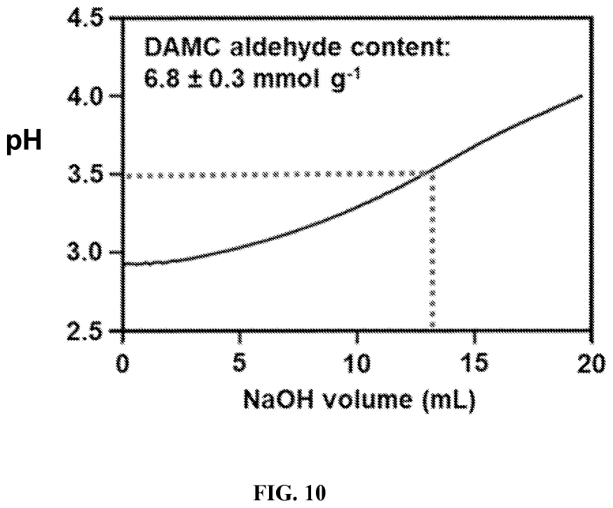

Linker Synthesis and Characterizations: The linker, an integral component of the nanocomposite hydrogels responsible for imparting strain-stiffening and self-healing properties, was synthesized and characterized in terms of functional groups (specifically aldehyde and carboxylate groups), particle size, and morphology. FIG. 9 shows linker synthesis steps, involving the oxidation of cellulose fibrils and DAMC. Via the controlled partial oxidation of cellulose fibrils using NaIO₄, DAMC was yielded and used as an intermediate precursor for linker synthesis. FIG. 10 shows a representative pH titration curve for DAMC fibrils. The NaOH amount required to neutralize the HCl released from the NH₂OH·HCl-aldehyde reaction, is used to measure the aldehyde content (6.8±0.3 mmol g⁻¹).

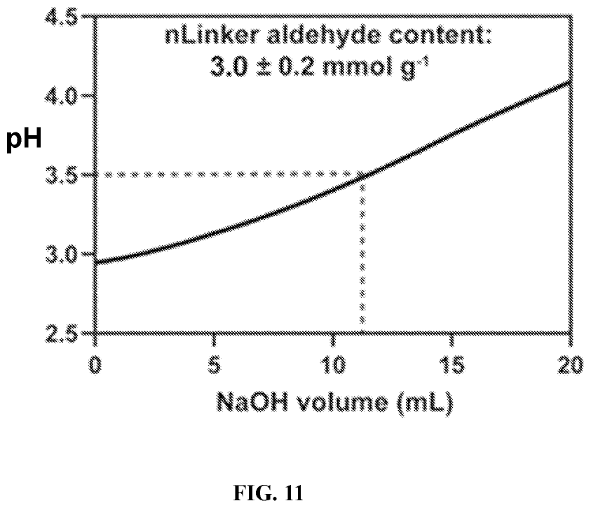

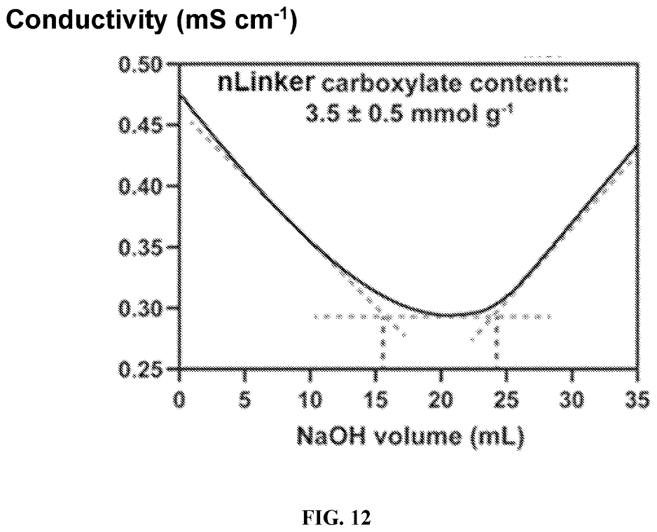

After subjecting DAMC to a controlled chlorite oxidation reaction, whereby almost half of the aldehyde groups were transformed to carboxylate groups, the bifunctional linker was isolated following successive thermal treatment and ethanol-mediated precipitation. FIGS. 11 and 12 show the representative titration curves of linker, bearing 3.0±0.2 mmol g⁻¹ aldehyde and 3.5±0.5 mmol g⁻¹ carboxylate groups, respectively. The coexistence of carboxylate and aldehyde groups enables concurrent physical (ionic) and chemical (dynamic covalent) bond formation with DH-ALG.

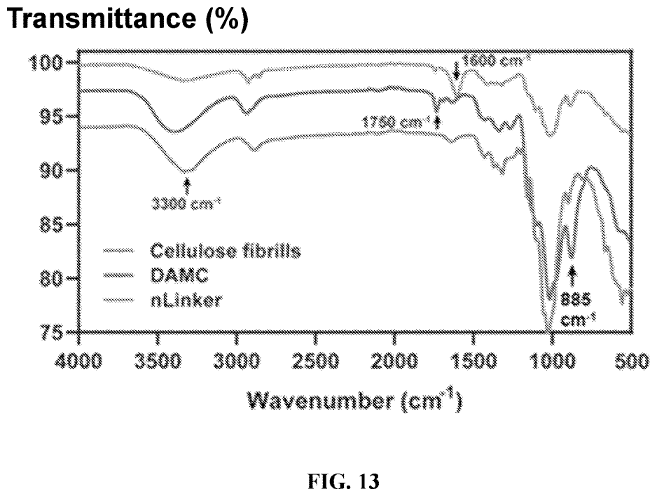

FIG. 13 shows the ATR-FTIR spectra of cellulose, DAMC fibrils, and the linker. In the DAMC spectrum, an increased intensity of the peak at ˜885 cm⁻¹ compared with cellulose was correlated to the hemiacetal linkages, arising from the interactions of aldehyde groups with the neighboring hydroxyl groups. The small peak at ˜1750 cm⁻¹ and broad peak at ˜3300 cm⁻¹ were assigned to the stretching vibrations of carbonyl and hydroxyl groups, respectively. In the linker spectrum, the aldehyde and carboxylate groups were confirmed via the stretching vibrations of carbonyl groups at ˜1750 cm⁻¹ and ˜1600 cm⁻¹, respectively.

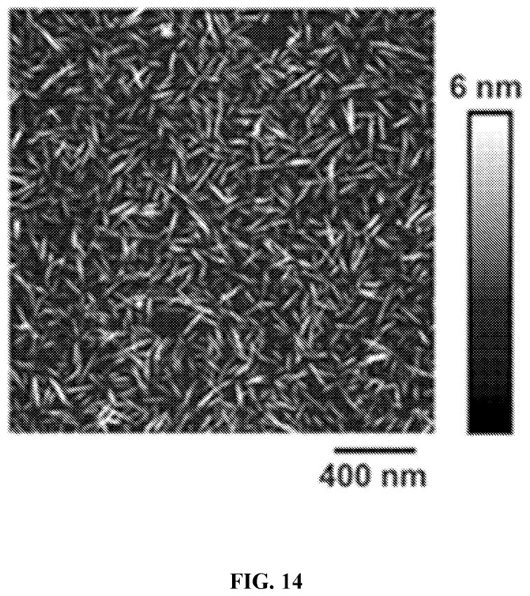

AFM images (see, for example, FIG. 14) showed that the linker is a rod-shaped nanoparticle with a length of ˜103±24 nm and a width of 5±2 nm. Hydrodynamic equivalent size of the linker was 132±11 nm at pH˜6 and ionic strength ˜3.5 mM, which was consistent with the previously reported values for bifunctional HCNC (˜108 nm) and trimmed anionic HCNC (˜140 nm, carboxylate content ˜3.4 mmol g⁻¹). The ζ-potential of linker at the same condition was −31±6 mV, confirming the negatively charged carboxylate groups.

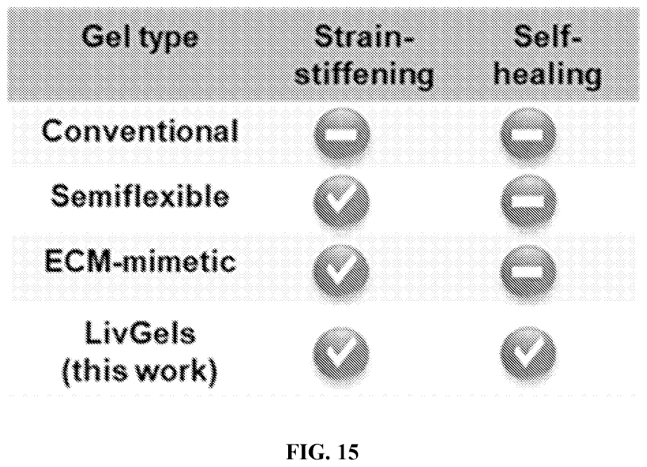

Mechanical Characterization of Hydrogels: An overview of the strain-stiffening and self-healing properties of common gels in the literature is provided in FIG. 15. Conventional gels comprising crosslinked, flexible polymers are typically inert to shear strain. However, gels made of semiflexible polymers, regardless of their biological or synthetic nature, may be shear-stiffening, albeit without self-healing. Similar to semiflexible polymer gels, ECM-mimetic analogs have been designed not only to undergo stiffening under strain, but also to enable other tissue-mimetic behaviors, such as anisotropic mechanics and shear-thinning. We aim to develop all-biopolymer-based nanocomposite hydrogels that have both strain-stiffening and self-healing properties and enable the adjustment of stiffness and stiffening response.

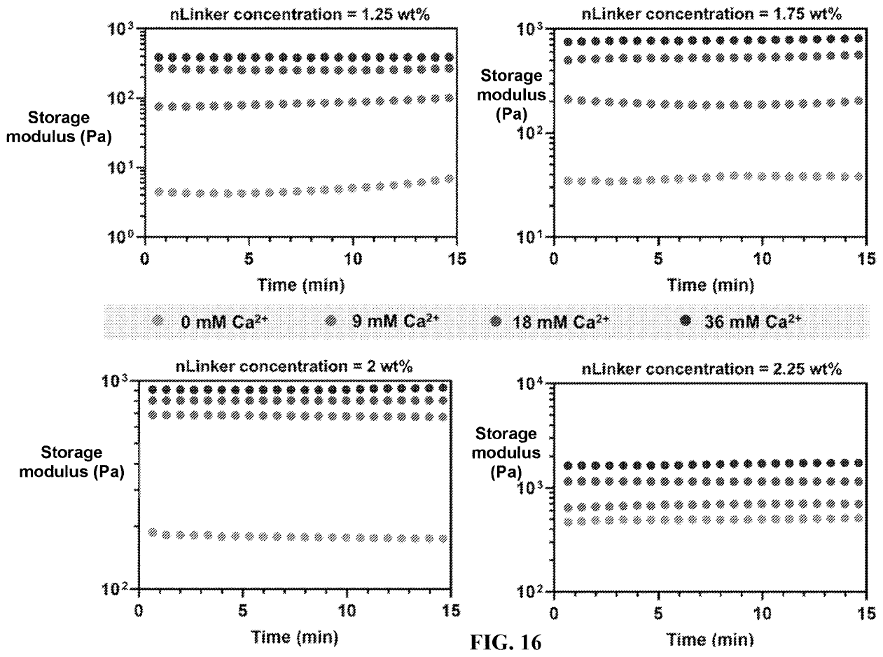

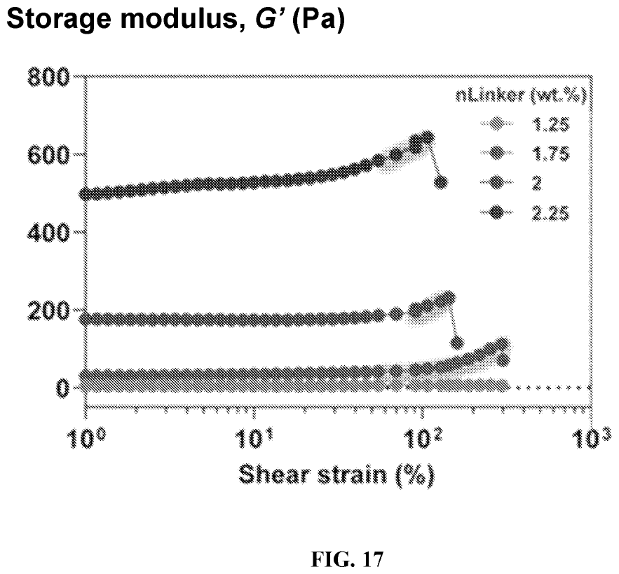

Before conducting mechanical characterizations, low-amplitude oscillatory rheology at a constant frequency of 1 rad s⁻¹ and strain of 0.1% was performed on gels ˜5 min after preparation, to examine their rheological behavior over time, as shown in FIG. 16. G′ did not significantly change within 15 min after hydrogel formation at the experimented linker or Ca²⁺ concentrations, indicating no significant evolution in network formation. To investigate the effect of linker-mediated dynamic hydrazone bonds on the linear and nonlinear mechanical properties of hydrogels, oscillatory strain amplitude sweep tests were conducted. FIG. 17 shows the G′ of hydrogels versus shear strain at varying linker concentrations. At a linker concentration of 1.75 wt. %, 2 wt. %, or 2.25 wt. %, the hydrogels underwent stiffening in the strain range of ˜56±28% to 277±54%, ˜66±10% to 188±88%, or ˜54±1% to 95±6% (n=3), respectively, yielding hydrogels. To determine the stiffening range of the hydrogels, we considered the stiffening onset as the strain point at which a 2% increase in the G′ occurred. The G′ of LivGels, containing 1.75 wt. %, 2 wt. %, or 2.25 wt. % linker, in the linear viscoelastic region (LVR) at a constant strain of 1%, was 30±15 Pa, 165±19 Pa, or 447±119 Pa (n=3), respectively, falling in the stiffness range of reconstituted ECM (G′˜30-1400 Pa). Type-1 collagen (G′˜30-1000 Pa), fibrin (G′˜300 Pa), and reconstituted basement membrane matrix (G′˜60-1400 Pa) are examples of reconstituted ECM.

In the hydrogels, the linkers form dynamic hydrazone bonds via a Schiff base reaction between the linker aldehyde and ALG-DH hydrazide groups. By increasing the linker concentration from 1.75 wt. % to 2.25 wt. % at a constant ALG-DH concentration (2 wt. %), the G′ increased in the LVE region and hydrogels became stiffer as a result of the increased number of hydrazone bonds and linker-mediated network reinforcement. The mechanical properties of the hydrogels strongly depend on the connectivity between polymer networks and particles. The hydrogel G′ within the LVR was used to estimate the average pore size (ξ)≈(G′/k_{B}T)^{-1/3}, where k_{B} is the Boltzmann constant, and Tis the absolute temperature. FIG. 18 shows that the hydrogel pore size monotonically decreased from 52±6 nm to 21±1 nm when the linker concentration increased from 1.75 wt. % to 2.25 wt. %, implying that the crosslinking density increased. Importantly, when the linker concentration ≤1.25 wt. %, no hydrogel was formed. The molar ratio of linker aldehyde groups to DH-ALG (2 wt. %) hydrazide in the hydrogel containing 1.25 wt. % linker was 1:0.4, which might not provide a sufficient number of dynamic hydrazone bonds for gel formation.

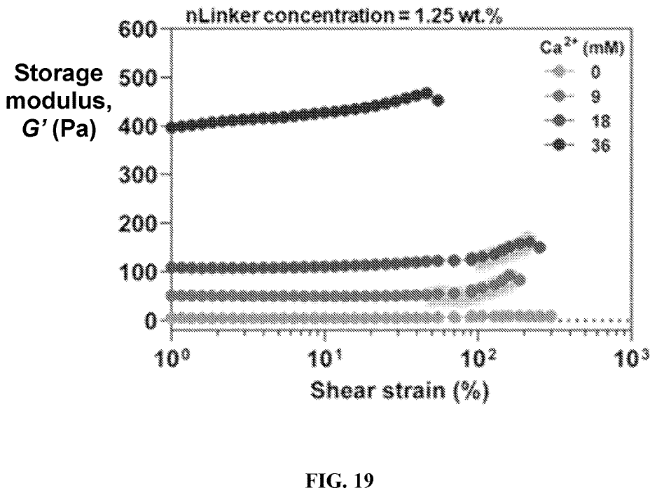

The effects of Ca²⁺-mediated ionic crosslinking along with the dynamic covalent bonds on the linear and non-linear mechanics of the hydrogels are presented in FIGS. 19-22. Note that 9 mM, 18 mM, or 36 mM of Ca²⁺ correspond to the saturation of ˜25%, ˜50%, or ˜100% of linker carboxylate groups, respectively. FIG. 19 shows the G′ and stiffening response of hydrogels, containing 1.25 wt. % linker and varying Ca²⁺ concentrations. The linker (1.25 wt. %) loaded with 9 mM or 18 mM of Ca²⁺ formed hydrogels with a non-linear stiffening behavior in the strain range of ˜59±9% to 242±33% or ˜91±1% to 188±28% (n=3), respectively. However, hydrogels containing the linker loaded with 36 mM of Ca²⁺ underwent stiffening over the whole strain range until the network was disrupted at a high strain. Increasing the Ca²⁻ concentration may shrink linker polymer chains via the neutralization of negatively charged carboxylates (stoichiometrically, at 36 mM, linker COO⁻:Ca²⁺=2:1 mol mol⁻¹), resulting in an increase in crosslinking density and G′. In another study, the addition of salt to the ALG-chitosan complexes increased G′, which was attributed to the loss of water as the charges were screened by the counterions. Datapoints at which the gels undergo a significant drop in G′ have not been presented in our figures. By increasing the Ca²⁺ concentration from 0 to 36 mM, the hydrogel stiffness increased because of the additional ionic bond formation within the hydrogel network. The G′ of hydrogel, containing linker (1.25 wt. %) with 9 mM or 18 mM of Ca²⁺, was 44±6 Pa or 94±20 Pa (within LVR, at strain ˜1%), respectively, mimicking the stiffness of type-1 collagen or reconstituted basement membrane matrix.

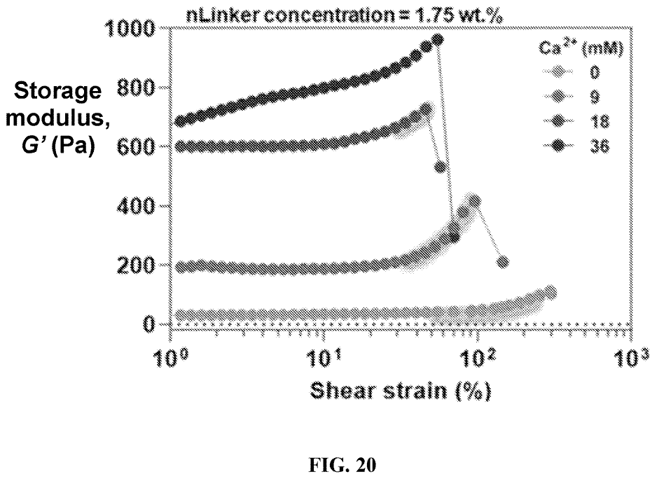

FIG. 20 presents the G′ and stiffening of hydrogels, containing 1.75 wt. % linker and varying Ca²⁺ concentrations. These gels, containing 9 or 18 mM of Ca²⁺, had a strain-stiffening behavior in the strain range of ˜58±31% to 164±62% or ˜31±8% to 71±26% (n=3), respectively. Compared with the hydrogels containing 1.25 wt. % linker with 9 mM or 18 mM Ca²⁺, these gels started stiffening at lower strains. The stiffness of hydrogels, containing 1.75 wt. % linker with 9 or 18 mM Ca²⁺, was 155±43 Pa or 508±138 Pa (at strain ˜1%, LVR), respectively, close to that of reconstituted ECM. Besides increasing hydrogel stiffness by increasing the Ca²⁺ concentration (0 to 36 mM) at a constant linker concentration, increasing the Ca²⁺-loaded linker from 1.25 wt. % to 1.75 wt. % resulted in stiffer hydrogels, implying the increased crosslinking density.

The strain-stiffening mechanism of hydrogels can be explained by stress localization at the hairy junction points where the majority of dynamic and ionic crosslinks exist. When the strain increases in the hydrogels, stress is applied to the hairs. As a result, the linker can re-orient locally along the strain direction, stretching the entire network and causing a stiffening response. Followed by linker reorientation, their extremely stiff crystalline body (Young's modulus=120-135 GPa) may be connected to each other via the protruding hairs, forming semiflexible fibrils with finite bending stiffness. At an extremely large strain, some fraction of hairy junction points may break, initiating the yield (failure) process.

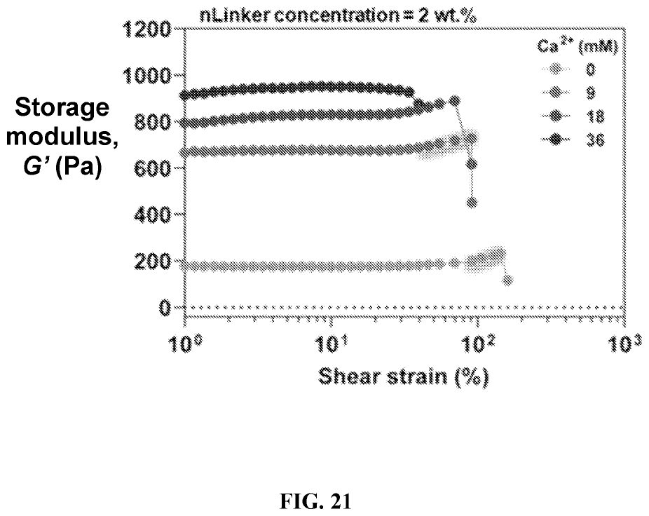

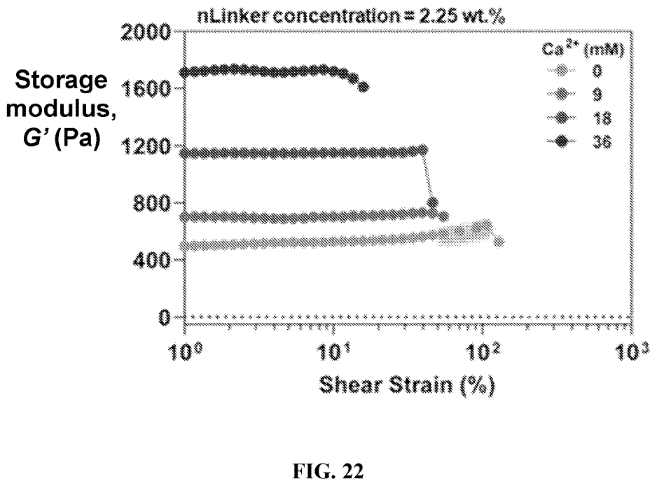

FIGS. 21 and 22 show the G′ and stiffening response of hydrogels, containing 2 wt. % and 2.25 wt. % linker at varying Ca²⁺ concentrations, respectively. The hydrogels without Ca²⁺ (at 2 or 2.25 wt. % linker) or with 9 mM of Ca²⁺ (at 2 wt. % linker) had a stiffening behavior, shown in shades; however, at higher Ca²⁺ concentrations (18 and 36 mM), the hydrogels did not have a non-linear response. At a constant Ca²⁺ concentration (9 mM), hydrogels with 2.25 wt. % linker showed no stiffening, while the analogs with 2 wt. % linker underwent ˜5% increase in G′, indicating that ionic crosslinking via linker further tailors the stiffening behavior. Additionally, the stiffness of hydrogels, containing 2 wt. % linker without Ca²⁺ (165±19 Pa) or with 9 mM Ca²⁺ (592±128 Pa), were in the range of reconstituted ECM. When the Ca²⁺ concentration was significantly high (36 mM), all the hydrogels containing 1.25 wt. % to 2.25 wt. % linker did not have any non-linear response.

At any linker concentration, Ca²⁺ increased the hydrogel stiffness as a result of ionic crosslinking, reducing the non-linear stiffening capability. At high Ca²⁺ concentrations (e.g., 18 mM for the hydrogels containing 2 or 2.25 wt. % linker and 36 mM for all the hydrogels), the loss of strain-stiffening behavior in the gels may be a result of increased network connectivity beyond the isostatic threshold, rendering the otherwise semi-flexible networks stiff. This may impair network fluctuations through changes in the linker-linker or DH-ALG-liner connections along the strain direction. Thus, the stiffening mechanism is mainly filament stretching (G∝σ^{1/2}). In contrast, if the average connectivity is below the isostatic threshold, the network is floppy and the deformation is through changes in the angle between the rods (G∝^{3/2}), allowing the alignment of rods along the strain direction.

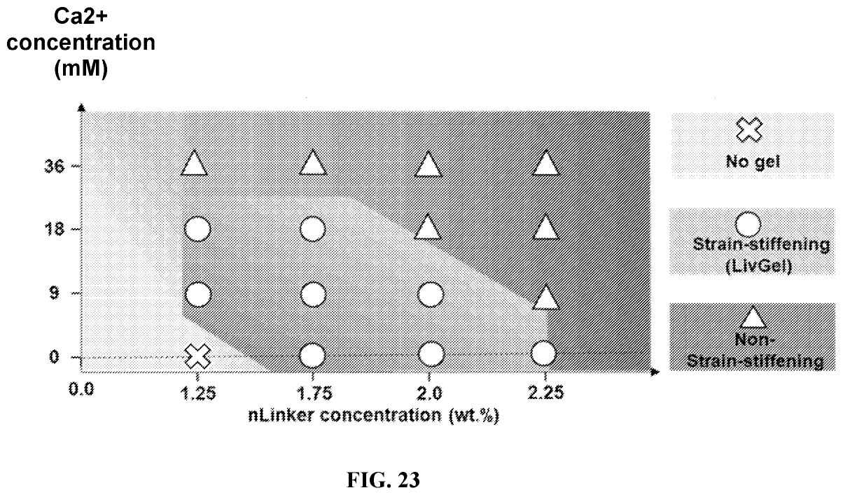

FIG. 18 shows the estimated hydrogel pore size at varying linker and Ca²⁺ concentrations. When the hydrogels, containing 1.25 wt. % linker, were ionically crosslinked with 9 mM of Ca²⁺, the pore size was 43.5±3 nm, which significantly decreased to 34±2 nm at 18 mM of Ca²⁺ and to 22±1 nm at 36 mM of Ca²′. In the hydrogels, containing 1.75 wt. % linker, when the Ca²⁺ concentration increased from 0 to 9 mM, the pore size significantly decreased from 52±6 nm to 27±1 nm, and the pore size changes were insignificant by increasing the Ca²⁺ concentration from 18 mM (19±1 nm) to 36 mM (18±1 nm). The evolution of pore size suggests that the maximum crosslinking via ionic bonding in the hydrogel matrix was obtained at ˜18 mM of Ca²⁺. Stoichiometrically, 32 mM of Ca²⁺ is required for the saturation of all the carboxylate groups (3.2±0.3 mmol g⁻¹) of DH-ALG, considering that 1 mol of Ca²⁺ binds to 2 mol of COO⁻. However, not all the linker loaded with Ca²⁺ may be available for ionic interactions with DH-ALG carboxylate groups, likely as a result of Ca²⁺-mediated internal crosslinking of carboxylates within the linker. In the hydrogels containing 2 wt. % linker (FIG. 18), the pore size significantly decreased from 28±1 nm (Ca²⁺ concentration=0) to 17±1 nm at 9 mM of Ca²⁺, followed by no significant changes as Ca²⁺ concentration was increased up to 36 mM. Similarly, increasing the Ca²⁺ concentration no longer affected the pore sizes of hydrogels, containing 2.25 wt. % of linker. FIG. 23 shows the phase diagram of hydrogels, including strain-stiffening (hydrogels) and non-strain-stiffening gels yielded at varying linker and Ca²⁺ concentrations. At a low (e.g., 1.25 wt. %) linker concentration, no hydrogel was formed without Ca²⁺-mediated ionic crosslinking as a result of insufficient hydrazone bonds. As the Ca²⁻-free linker concentration increased up to 2.25 wt. %, hydrogels were formed. At any constant linker concentration ≤2 wt. %, increasing Ca²⁺ concentration initially yielded hydrogels, followed by non-strain-stiffening gels. At 2.25 wt. % of linker, Ca²⁺ rendered the hydrogels non-strain-stiffening. Accordingly, the hydrogels are converted at a specific concentration range of linker and Ca²⁺.

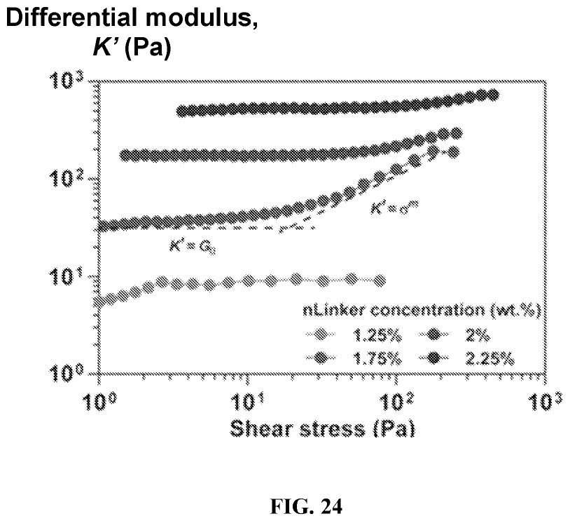

Quantitative Analysis of Strain-Stiffening Behavior: To quantify stiffening, differential shear modulus, K′=∂σ/∂γ, where σ and γ are shear stress and strain, respectively, was calculated for the hydrogels, containing varying linker and Ca²⁺ concentrations. FIG. 24 shows K′ versus shear stress at a constant frequency for hydrogels, containing varying Ca²⁺-free linker concentrations. A linear region at low stress, followed by a nonlinear region at higher stress were observed at any linker concentration, except when linker concentration=1.25 wt. % at which no hydrogel was formed. In the lower stress region, K′ was equal to G′ in the LVR (G₀), whereas in the high stress region, K′ had an exponential correlation with shear stress (K′∂σ′″). The stiffening index (m) and critical stress (θ_{c}) are indicators of stiffening intensity and the onset of nonlinear region, respectively. When m is close to 0.5, the dominant stiffening mechanism is filament stretching; however, as m approaches 1.5, the network fluctuation mainly regulates the stiffening responses. By conducting oscillatory amplitude sweep tests and extracting the exponents, we investigated how the hydrogel components, specifically linker or Ca²⁺-loaded linker, and the corresponding networks affect the transition between the stiffening mechanisms (i.e., network fluctuation versus single filament stretching) and control the dominant power-law scaling behavior.

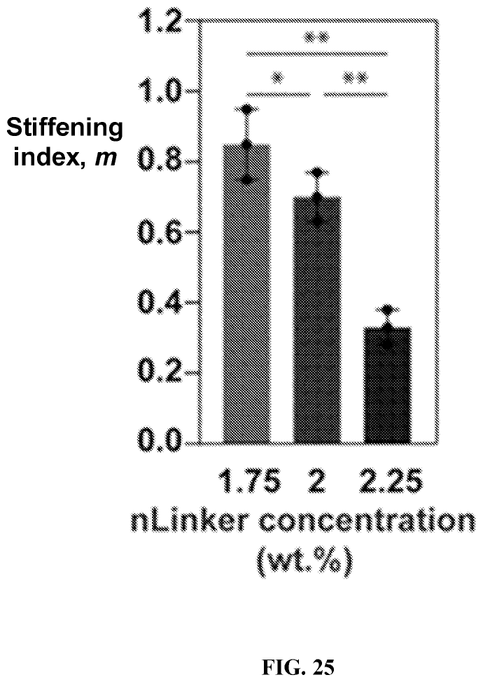

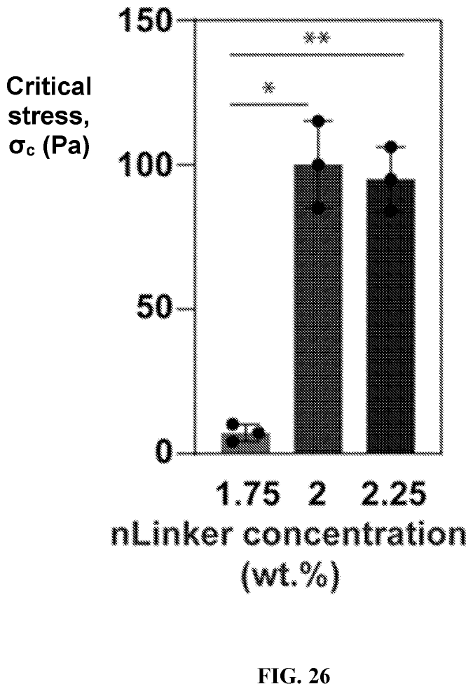

Based on the K′ plots (FIG. 24), the m and σ_{c} values for hydrogels containing varying concentrations of Ca²⁺-free linkers were obtained, which are presented in FIGS. 25 and 26, respectively. The m values for hydrogels containing 1.75, 2, and 2.25 wt. % of linker were 0.85±0.10, 0.72±0.07, and 0.30±0.11, respectively, indicating a decrease in the stiffening extent by increasing the linker concentration. The m values imply that the dominant stiffening mechanism is a combination of filament stretching and network fluctuations at low linker concentrations, which shifts to more filament stretching by increasing the linker concentration (≥2 wt. %). The σ_{c} values of hydrogels, containing 1.75, 2, or 2.25 wt. % linker were 7±3, 100±15, or 95±11 Pa, respectively, showing a significant increase in σ_{c}, i.e., less non-linear responsiveness, by increasing the linker concentration. The decrease in m and increase in σ_{c} may be associated with an increase in the stiffness (G₀) by increasing the crosslinking degree via the formation of hydrazone bonds and/or the reinforcing contribution of linker, rendering the hydrogels less dynamically responsive to the applied strain. These results show that by changing the linker concentration, the nonlinear mechanical response of hydrogels is readily tailored at a constant matrix biopolymer (DH-ALG) concentration (2 wt. %).

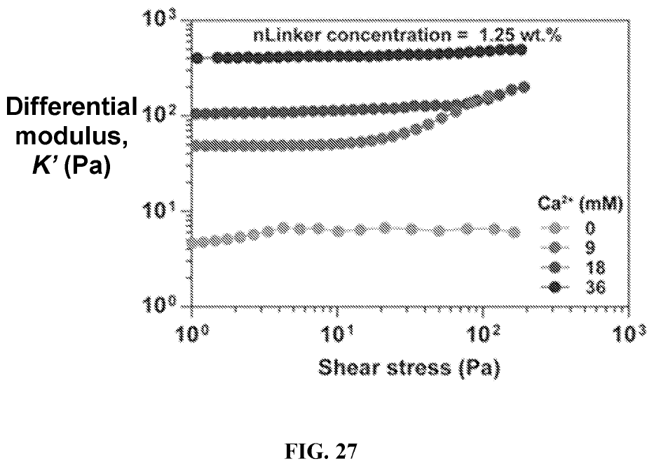

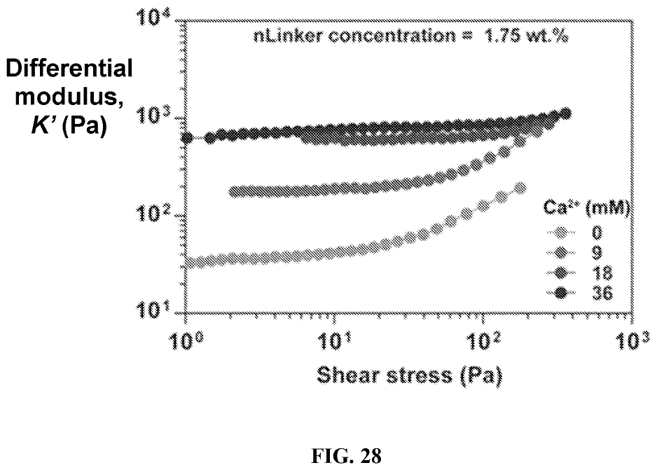

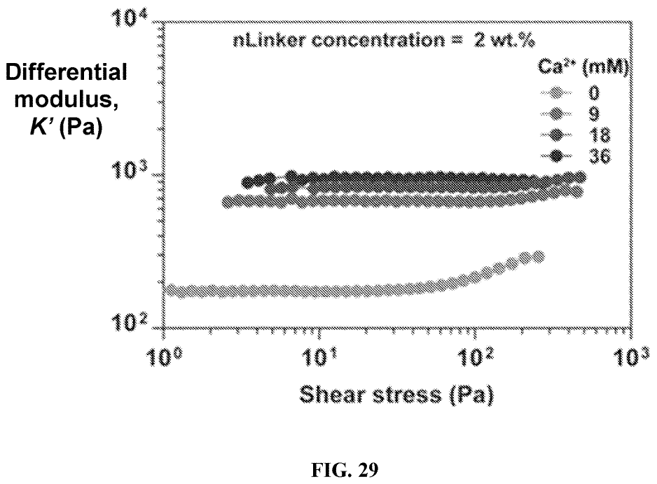

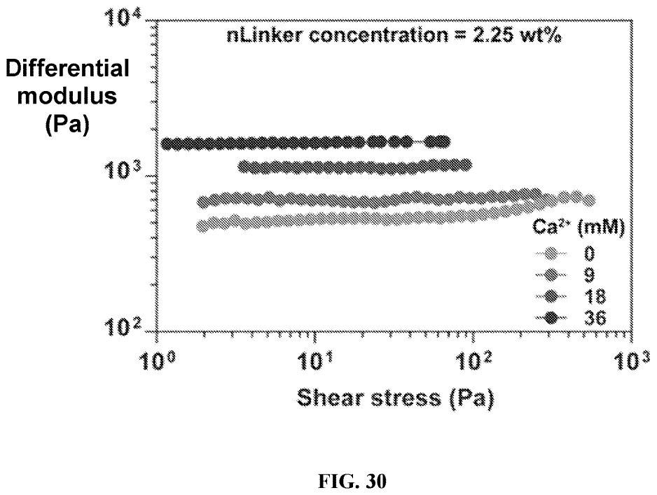

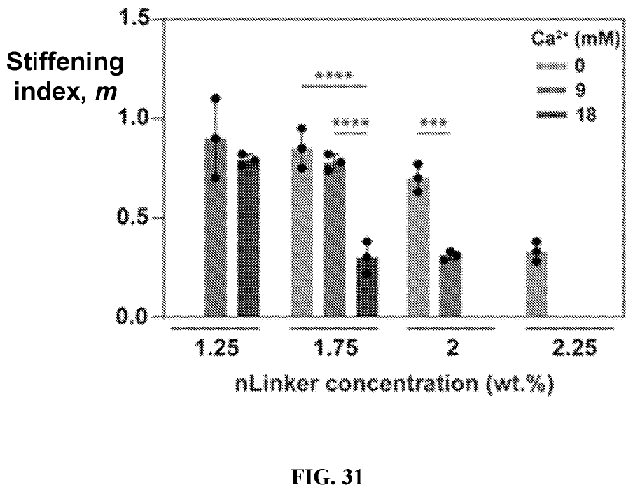

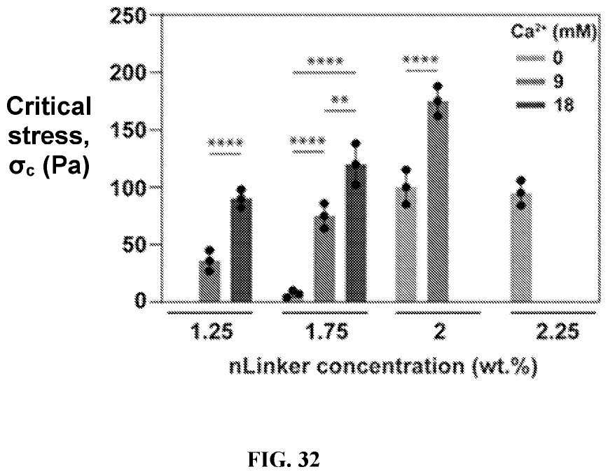

To quantify the effect of Ca²⁺ on the strain-stiffening behavior of hydrogels, the K′ of hydrogels, containing 2 wt. % of DH-ALG and varying Ca²⁺ concentrations, was measured as a function of oscillatory shear stress at a constant linker concentration of 1.25, 1.75, 2, or 2.25 wt. %, as presented in FIGS. 27-30, respectively. For the hydrogels containing 1.25 wt. % or 1.75 wt. % of linker loaded with 9 or 18 mM Ca²⁺, or 1.75 wt. % or 2 wt. % of Ca²⁺-free linker, as well as the hydrogels containing 2 wt. % of linker loaded with 9 mM Ca²⁺, a linear region at low stress and a non-linear region at higher stress were observed, which were consistent with the oscillatory strain sweep results. For all the hydrogels, G₀ at low stress increased by increasing the Ca²⁺ concentration from 0 mM to 36 mM, confirming an increase in the ionic crosslinking density. The m and σ_{c} values of hydrogels containing varying Ca²⁺ concentrations are shown in FIGS. 31 and 32, respectively. The m values for hydrogels, containing 1.25 wt. % linker loaded with 9 or 18 mM Ca²⁺ were ˜0.87±0.18 or ˜0.73±0.03, respectively. For these gels, by increasing the Ca²⁺ concentration, the σ_{c} value shifted significantly from 28±11 Pa at 9 mM of Ca²⁺ to 90±8 Pa at 18 mM of Ca²⁺. Similarly, in the hydrogels containing 1.75 wt. % or 2 wt. % linker, by increasing the Ca²⁺ concentrations, m decreased and σ_{c} increased. Such reduction in the mechanical responsiveness may be attributed to an increase in hydrogel stiffness as a result of dual dynamic hydrazone/ionic crosslinking and the increased network connectivity beyond the isostatic threshold, rendering the semi-flexible networks stiff. Accordingly, when the hydrogels, containing 2.25 wt. % of linker were loaded with Ca²⁺ (FIG. 30), they did not have any strain-stiffening behavior. Additionally, the m values of hydrogels, containing 1.75 wt. % of linker, imply that the stiffening mechanism in the absence of Ca²⁺ (m=0.85±0.10) may be a result of filament stretching and network fluctuations, whereas by increasing the Ca²⁺ concentrations (e.g., m=0.30±0.08 at 18 mM), the stiffening may be regulated by filament stretching. A reason for this transition may be a high density of ionic crosslinking by which the local reorientation (bending) of linkers along the strain direction is restricted and therefore the stiffening is governed via filament stretching.

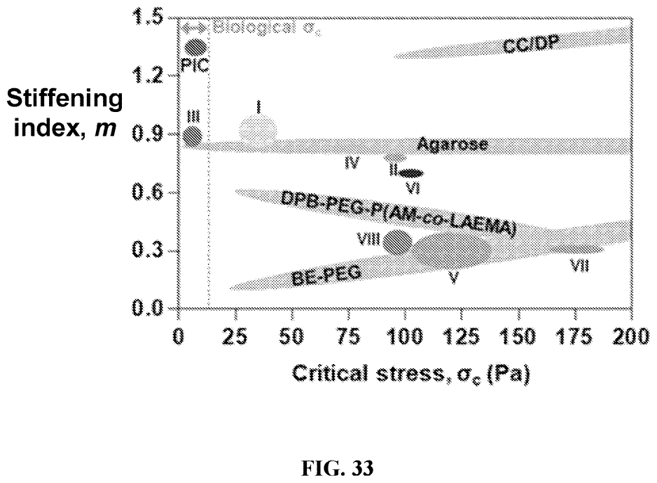

FIG. 33 summarizes the stiffening parameters (m and σ_{c}) of biological and synthetic gels, as well as the inventive nanocomposite hydrogels. The hydrogels containing Ca²⁺-free linker (1.75 wt. %) were engineered to resemble the strain-stiffening characteristics of biological gels (e.g., 0.5 Pa<σ_{c}<10 Pa). In the stress-stiffening region, m has been predicted to be 1.5 for a single semiflexible polymer, as observed in fibrin fibers and actin filaments. Among the developed hydrogels, synthetic polyisocyanides (PIC) gels have comparable strain-stiffening parameters with biological analogs. Synthetic boronate ester-crosslinked 4-arm polyethylene glycol (BE-PEG) hydrogels have strain-stiffening properties with σ_{c} ranging from 20 Pa to 1000 Pa and m ranging from 0.1 to 0.5, regulated by the polymer concentration, temperature, and pH. Agarose hydrogels are a class of ECM-like biomaterials with strain-stiffening behavior because of their semi-flexible nature. The hydrogels offer a broad range of m, spanning from ˜0.87 (9 mM Ca²⁺-loaded linker concentration=1.25 wt. %) to ˜0.25 (Ca²⁺-free linker concentration=2.25 wt. %), with σ_{c} ranging from ˜28 Pa to ˜157 Pa, adjusted by the crosslinking density or modes (i.e., hydrazone and ionic bonding). Diphenylboronic acid-terminated telechelic poly-(ethylene glycol)-poly(acrylamide-co-2-lactobionamidoethylmethacrylamide) (DPB-PEG-P(AM-co-LAEMA)) and O-carboxymethyl chitosan-dibenzaldehyde-terminated telechelic-ploy-(ethylene glycol) (CC/DP) are other hydrogels with stiffening responses that are mechanically mimicked by the hydrogels. Note that DPB-PEG-P(AM-co-LAEMA) and CC/DP gels do not recapitulate the stiffening properties, specifically σ_{c}, of biological gels. Overall, the inventive hydrogels provide a novel bio-based platform that mimic the dynamic mechanical properties of ECM.

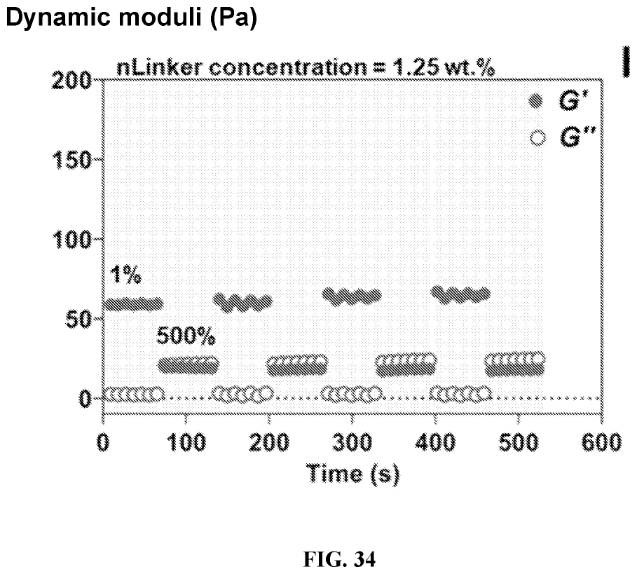

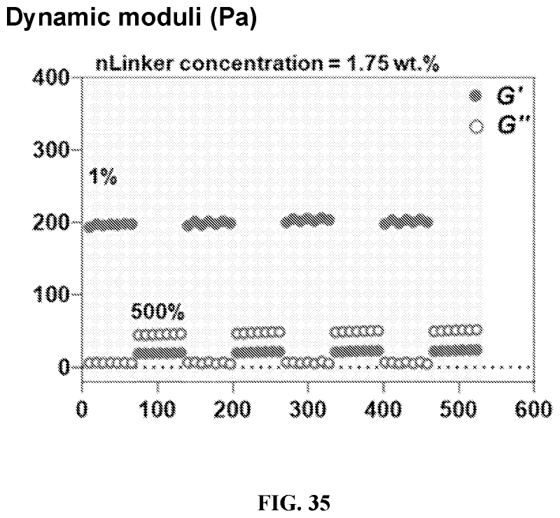

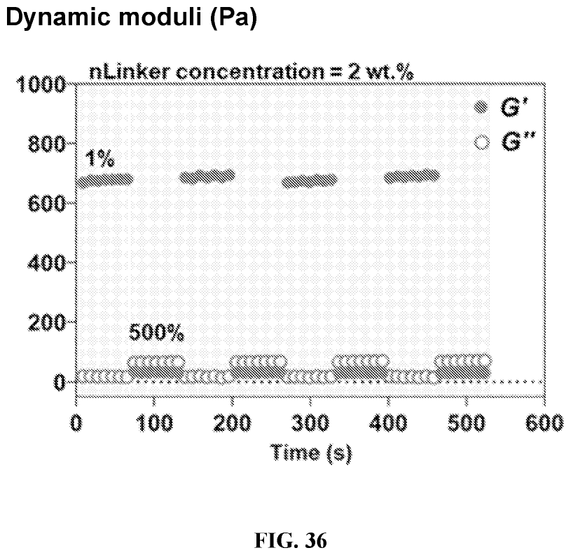

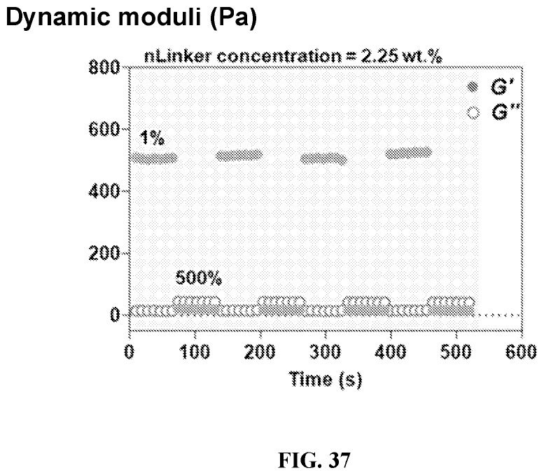

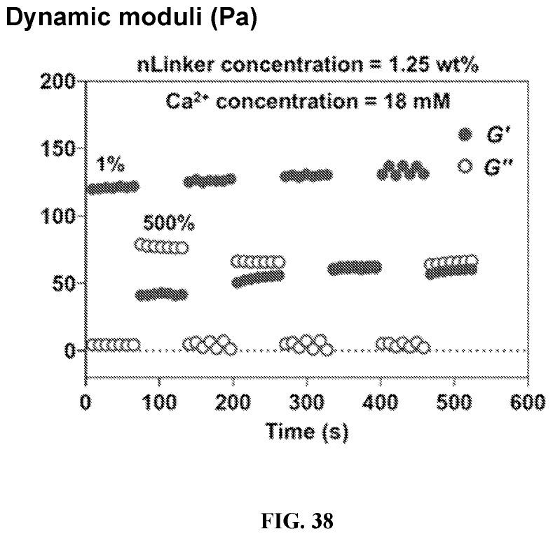

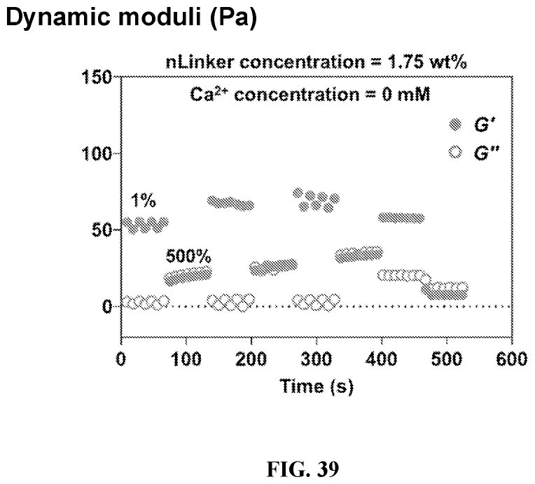

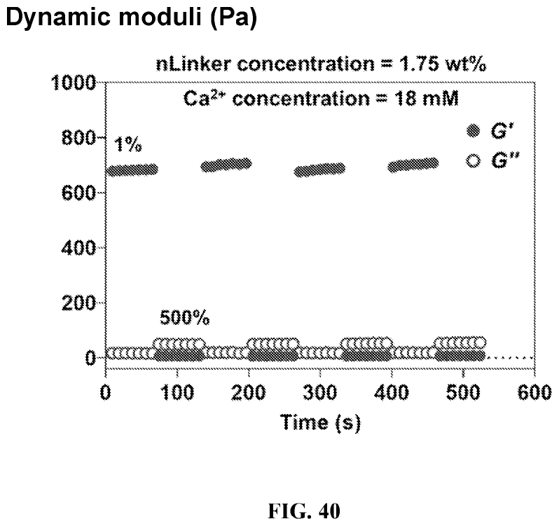

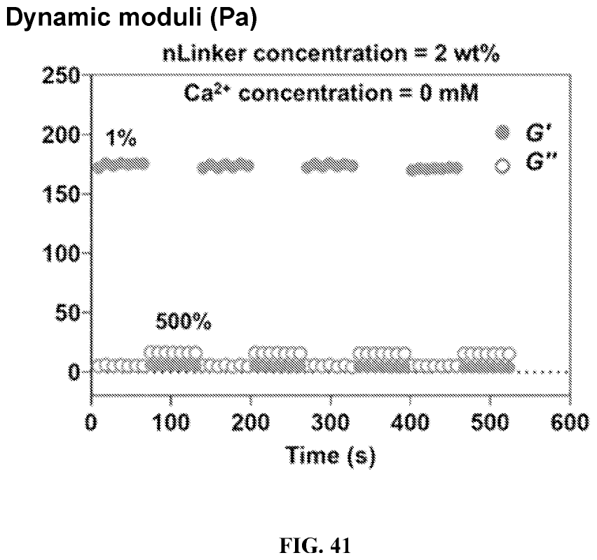

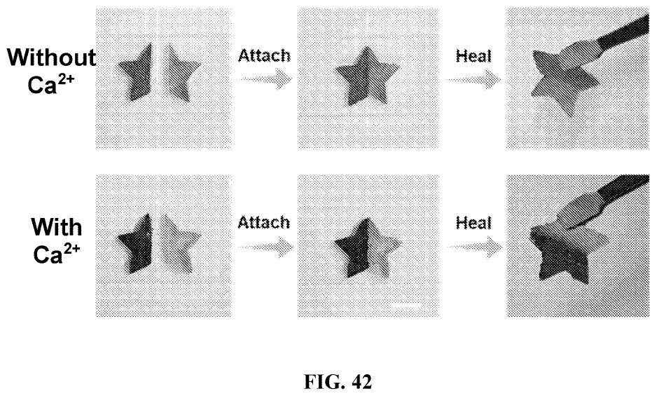

Qualitative and Quantitative Analyses of Nanocomposite Hydrogel Self-Healing Properties: The dynamic covalent hydrazone bonds and physical ionic interactions have been adopted extensively to develop self-healing gels. Accordingly, the developed hydrogels were engineered using aldehyde- and carboxylate-bearing linker to include reversible bonds that would enable self-healing behavior when mixed with DH-ALG. The self-healing capability of the hydrogels was assessed by strain amplitude measurements in which repeating cycles of low (1%) and high (500%) strains were used. FIGS. 34-37 present the recovery tests in which the dynamic moduli of hydrogels, containing varying linkers concentrations loaded with 9 mM of Ca²⁺, are registered over time. The recovery tests of other gels, containing varying linker and Ca²⁺ concentrations, are shown in FIGS. 38-41. These experiments were conducted to investigate how rapidly the nanocomposite hydrogels recover their solid-like structure after the elimination of high strain. By applying strain=500%, the nanocomposite hydrogels had a fluid-like (viscous) behavior, reflected in G′<G″. Right after each cycle of high strain, the nanocomposite hydrogels recovery was monitored at 1% strain and 1 rad s⁻¹ angular frequency. All strain-stiffening hydrogels, containing either Ca²⁺-free linker (formed via hydrazone bonds) or Ca²⁺-loaded linker (formed via both ionic interactions and hydrazone bonds), had excellent recovery within seconds even after 4 cycles. At an extremely large strain, a fraction of hairy junctions/crosslink points between linker/linker or linker/DH-ALG may break. These broken junction points, made of reversible bonds, reform upon strain cessation, healing the structure of hydrogels. FIG. 42 shows a qualitative self-healing assessment of the nanocomposite hydrogels, containing Ca²⁺-free linker (2 wt. %) or 9 mM Ca²⁺-loaded linker (2 wt. %). The nanocomposite hydrogels were prepared in star-shape molds, cut in two pieces, and healed in 10 min after juxtaposing the cut interfaces. The healed hydrogels, containing Ca²⁺-free linker or Ca²⁺-loaded linker, were mechanically robust and preserved their integrity upon hanging.

It should be understood that modifications to the embodiments disclosed herein can be made to meet a particular set of design criteria. For instance, the number of components or parameters can be varied to meet a particular objective. As another example, component configurations may be varied to meet a particular set of design criteria.

It will be apparent to those skilled in the art that numerous modifications and variations of the described examples and embodiments are possible in light of the above teachings of the disclosure. The disclosed examples and embodiments are presented for purposes of illustration only. Other alternative embodiments may include some or all of the features of the various embodiments disclosed herein. For instance, it is contemplated that a particular feature described, either individually or as part of an embodiment, can be combined with other individually described features, or parts of other embodiments. The elements and acts of the various embodiments described herein can therefore be combined to provide further embodiments.

It is the intent to cover all such modifications and alternative embodiments as may come within the true scope of this invention, which is to be given the full breadth thereof. Additionally, the disclosure of a range of values is a disclosure of every numerical value within that range, including the end points. Thus, while certain exemplary embodiments of the apparatus and process and/or utilization and methods of making and using the same have been discussed and illustrated herein, it is to be distinctly understood that the invention is not limited thereto but may be otherwise variously embodied and practiced within the scope of the following claims.

What is claimed is:

1. An acellular nanocomposite hydrogel comprising:
a biopolymer comprising a plurality of polymeric chains, wherein the polymeric chains have first active sites and second active sites; and
a plurality of nanoparticle linkers, wherein the linkers comprise cellulose bodies having a plurality cellulose chains protruding from their ends,
wherein a first portion of the cellulose chains are functionalized with first groups configured to bond to the first active sites, and
wherein a second portion of the cellulose chains are functionalized with second groups configured to bond to the second active sites.

2. The hydrogel of claim 1, wherein the first groups are configured to covalently bond with the first active sites, and the second groups are configured to ionically bond with the second active sites.

3. The hydrogel of claim 2, wherein the first groups are aldehyde groups and the first active sites are hydrazides, such that the aldehyde groups and the hydrazides covalently bond to crosslink the linkers with the biopolymer.

4. The hydrogel of claim 2, wherein the second groups are carboxylate groups and the second active sites are carboxylate sites, such that the carboxylate groups are configured to ionically bond to the carboxylate sites to crosslink the linkers with the biopolymer.

5. The hydrogel of claim 1, wherein a portion of the nanoparticle linkers form linker bridges including linkers crosslinked to one another via one or more cations.

6. The hydrogel of claim 5, wherein the one or more cations include calcium ions.

7. The hydrogel of claim 5, wherein the first groups are configured to covalently bond with the first active sites, and the cations and/or the second groups are configured to ionically bond with the second active sites.

8. The hydrogel of claim 7, wherein the first groups are aldehyde groups and the first active sites are hydrazides, such that the aldehyde groups and the hydrazides covalently bond to crosslink the linkers with the biopolymer.

9. The hydrogel of claim 7, wherein the second groups are carboxylate groups and the second active sites are carboxylate sites, such that the carboxylate groups and/or the cations are configured to ionically bond to the carboxylate sites to crosslink the linkers with the biopolymer.

10. The hydrogel of claim 1, wherein the hydrogel exhibits shear-stiffening and self-healing properties.

11. An acellular nanocomposite hydrogel comprising:
a dihydrazide-modified alginate biopolymer comprising a plurality of polymeric chains, wherein the polymeric chains have hydrazide active sites and carboxylate active sites; and
a plurality of nanoparticle linkers, wherein the linkers comprise cellulose bodies having a plurality cellulose chains protruding from their ends,
wherein a first portion of the cellulose chains are functionalized with aldehyde groups configured to bond to the hydrazide active sites, and
wherein a second portion of the cellulose chains are functionalized with carboxylate groups configured to bond to the carboxylate active sites.

12. The hydrogel of claim 11, wherein a portion of the nanoparticle linkers form linker bridges including linkers crosslinked to one another via one or more cations.

13. The hydrogel of claim 12, wherein the linker bridges are configured to ionically bond to the carboxylate sites to crosslink the linkers with the biopolymer.

14. A method of forming an acellular nanocomposite hydrogel, the method comprising:
forming a plurality of nanoparticle linkers by:
oxidizing cellulose bodies to provide dialdehyde modified cellulose bodies,
oxidizing the dialdehyde modified cellulose bodies to provide further modified cellulose bodies having a plurality cellulose chains protruding from their ends, wherein a first portion of the cellulose chains are functionalized with aldehyde groups, and a second portion of the cellulose chains are functionalized with carboxylate groups;
mixing the further modified cellulose bodies with an ionic solution such that the second portion of the cellulose chains are also functionalized with cations;
mixing the plurality of nanoparticle linkers with a biopolymer comprising a plurality of polymeric chains, having first active sites and second active sites such that the aldehyde groups bond to the first active sites and the carboxylate groups and/or cations bond to the second active sites.

15. The method of claim 14, wherein the aldehyde groups covalently bond with the first active sites, and the carboxylate groups and/or the cations ionically bond with the second active sites.

16. The method of claim 15, wherein the first active sites are hydrazides such that the aldehyde groups and the hydrazides covalently bond to crosslink the linkers with the biopolymer.

17. The method of claim 16, wherein the second active sites are carboxylate sites such that the carboxylate groups and/or the cations ionically bond to the carboxylate sites to crosslink the linkers with the biopolymer.

18. A method of regenerating tissue, the method comprising:
integrating an acellular nanocomposite hydrogel with a host tissue, wherein the hydrogel comprises
a biopolymer comprising a plurality of polymeric chains, wherein the polymeric chains have first active sites and second active sites; and
a plurality of nanoparticle linkers, wherein the linkers comprise cellulose bodies having a plurality cellulose chains protruding from their ends,
wherein a first portion of the cellulose chains are functionalized with first groups configured to bond to the first active sites, and
wherein a second portion of the cellulose chains are functionalized with second groups configured to bond to the second active sites.

19. The method of claim 18, wherein the first groups are configured to covalently bond with the first active sites, and the second groups are configured to ionically bond with the second active sites;
wherein the first groups are aldehyde groups and the first active sites are hydrazides such that the aldehyde groups and the hydrazides covalently bond to crosslink the linkers with the biopolymer; and
wherein the second groups are carboxylate groups and the second active sites are carboxylate sites such that the carboxylate groups ionically bond to the carboxylate sites to crosslink the linkers with the biopolymer.

20. The method of claim 19, wherein a portion of the nanoparticle linkers form linker bridges including linkers crosslinked to one another via one or more cations.
